(19) United States
(12) Patent Application Publication
(10) Pub. No.: US 2011/0003767 A1
(43) Pub. Date: Jan. 6, 2011

(54) Inhibitors of Acetyl-CoA Carboxylase for Treatment of Neuronal Hypometabolism

(75) Inventor: Samuel T. Henderson, Golden, CO (US)

Correspondence Address: SWANSON & BRATSCHUN, L.L.C., 8210 SOUTHPARK TERRACE, LITTLETON, CO, 80120, US

(73) Assignee: NEUERA PHARMACEUTICALS, INC., Broomfield, CO (US)

(21) Appl. No.: 12/599,476

(22) PCT Filed: May 14, 2008
(86) PCT No.: PCT/US08/06352

§ 371 Date: Nov. 9, 2009

Related U.S. Application Data
(60) Provisional application No. 60/917,886, filed on May 14, 2007.

(51) Int. Cl. A61K 31/7076; A61K 31/5377; A61K 31/4545; A61K 31/341; A61K 31/22; A61K 31/20; A61K 31/40; A61K 31/366; A61K 31/4188; A61P 25/28

(52) U.S. Cl. 514/47; 514/235.5; 514/316; 514/473; 514/546; 514/558; 514/428; 514/450; 514/387

(57) ABSTRACT

This invention relates to methods of using inhibitors of the enzyme acetyl-CoA carboxylase (ACC) for the treatment, prevention, inhibition or alleviation of diseases associated with neuronal hypometabolism and/or loss of cognitive function caused by reduced neuronal metabolism such as, for example, Age Associated Memory Impairment (AAMI), Mild Cognitive Impairment (MCI), Alzheimer's disease, Parkinson's disease, Friedreich's Ataxia (FRDA), GLUT1-deficient Epilepsy, Leprechaunism and Rabson-Mendenhall Syndrome, Coronary Arterial Bypass Graft (CABG) dementia, anesthesia induced memory loss, Huntington's disease and many others.

### FIELD OF THE INVENTION

This invention relates to the field of therapeutic agents for the treatment of Alzheimer's disease, Mild Cognitive Impairment, and other diseases associated with reduced neuronal metabolism, including Parkinson's disease, Huntington's Disease, and epilepsy.

### BACKGROUND OF THE INVENTION

Numerous diseases are associated with reduced neuronal glucose metabolism and a need exists to treat these conditions. The compounds of the present invention can be useful to treat these conditions.

Alzheimer's disease (AD) is a progressive neurodegenerative disorder that primarily affects the elderly. In 1984, Blass and Zemcov (Blass and Zemcov 1984) proposed that AD resulted from a decreased metabolic rate in sub-populations of cholinergic neurons. However, it has become clear that AD is not restricted to cholinergic systems, but involves many types of transmitter systems, and several discrete brain regions. The decreased metabolic rate appears to be related to decreases in glucose utilization. Brain imaging techniques have revealed decreased uptake of radiolabeled glucose in the brains of AD patients, and these defects can be detected well before clinical signs of dementia occur (Reiman, Caselli et al. 1996). Measurements of cerebral glucose metabolism indicate that glucose metabolism is reduced 20-40% in AD resulting in critically low levels of ATP.

The cause of the decreased glucose metabolism remains uncertain, but may be related to processing of the amyloid precursor protein (APP). Mutations that alter the processing of APP have been implicated in early onset AD. Early onset cases occur before the age of 60 and in many cases have been associated with mutations in three genes: APP, presenilin 1 (PS1) and presenilin 2 (PS2). Mutations in these genes lead to aberrant processing of the APP protein (for review see (Selkoe 1999)). Where examined, these pathological mutations result in early defects in cerebral glucose metabolism. Individuals harboring a double mutation at APP670/671 (the “Swedish mutation”) exhibit pathological decreases in glucose metabolism in temporal lobes, often before clinical manifestations of dementia are evident. Mice carrying an APP V717F transgene exhibit regional defects in cerebral glucose metabolism. Also, mutations in the presenilin genes may directly increase susceptibility to glucose deprivation.

Attempts to compensate for reduced cerebral metabolic rates in AD have met with some success. Elevation of serum ketone body levels in AD patients raises cognitive scores (Reger, Henderson et al. 2004). However, this reported method requires administration of large amounts of fat to generate the sufficient levels of ketone bodies. Therefore, a need exists for compounds that can elevate ketone levels without large fat consumption.

Parkinson disease (PD) is the second most common neurodegenerative disease after AD. PD is characterized by motor abnormalities, including tremors, muscle stiffness, lack of voluntary movements, and postural instability. A primary neuropathological feature of PD is the loss of dopaminergic neurons in the substantia nigra pars compacta (SNpc).

While the cause of sporadic PD is uncertain, several lines of evidence suggest that defects in oxidative phosphorylation may contribute to its pathogenesis. For example, 1-methyl-4-phenyl-1,2,3,6-tetrahydropyridine (MPTP), blocks complex I (NADH-ubiquinone oxidoreductase) of the mitochondrial electron transport chain, and causes typical symptoms of PD and the loss of dopaminergic neurons. Reduction in complex I activity has also been reported in PD tissues. This defect is not confined only to the brain but has also been found in platelets from PD patients.

D-β-Hydroxybutyrate (BHB) is a ketone body produced by hepatocytes and, to a lesser extent, by astrocytes. BHB acts as an alternative source of energy in the brain when glucose supply is limited such as during starvation. BHB has been found to protect from MPTP-related complex I inhibition, by enhancing oxidative phosphorylation (Tieu, Perier et al. 2003).

FRDA is a recessive disease characterized by progressive ataxia, hypertrophic cardiomyopathy, early onset of insulin-resistant diabetes, invalidism, and premature death. FRDA is a genetic disorder caused by a deficiency of frataxin, a 210 amino acid nuclear-encoded mitochondrial protein. Low levels of the protein are due to the expansion of an intronic GAA repeat, leading to decreased mRNA levels. FRDA patients show a decrease in the activity of the mitochondrial enzyme aconitase. Aconitase is responsible for conversion of citrate to isocitrate, the first step in the Krebs (also known as citric acid or TCA) cycle. Deficiency of frataxin in human patients is thought to lead primarily to defects in the TCA cycle. Recent work shows that elevation of blood ketone bodies, a normal response to fasting, can increase mitochondrial citrate and isocitrate levels, thus overcoming the block in aconitase found in FRDA. A ketone body-based therapy could provide an effective treatment for this group of patients.

GLUT1-deficient Epilepsy is characterized by infantile seizures, delayed development, and acquired microcephaly with mental retardation. GLUT1-deficient epilepsy results from several types of mutation in the gene of GLUT1. Glucose transporter 1 (GLUT1) is the major protein responsible for the transport of glucose from bloodstream into the brain. Under standard dietary conditions, the brain is almost entirely dependent upon blood glucose for energy. However, under some circumstances, such as starvation, ketone bodies can provide a source of energy different from glucose. Ketone bodies do not rely on GLUT1 for transport into the brain and therefore may provide energy in GLUT1-deficient syndrome. Ketone body therapy may therefore become a practical method for lifelong treatment of these patients.

Leprechaunism and Rabson-Mendenhall syndrome are rare disease characterized by insulin resistance, persistent hyperglycemia and retardation of growth. Subjects rarely survive past 20 years of age. These syndromes result from mutations in the insulin receptor gene, which lower the receptors affinity for insulin. The current treatment consists of administration of increasing doses of insulin (up to several thousand units per day). This treatment yields only weak results due to the poor binding of insulin to its receptor. Ketone bodies have been shown to mimic the effects of insulin's stimulation of the PDH multienzyme complex, thereby increasing the Krebs TCA cycle metabolite levels, increasing the energy output in the form of ATP, and enhancing metabolic efficiency. A ketone-rich, or ketogenic diet may prove an effective treatment of these conditions.

A great number of other diseases and syndromes are associated with decreased neuronal metabolism. Such conditions include Age Associated Memory Impairment (AAMI), Mild Cognitive Impairment (MCI), Coronary Arterial Bypass Graft (CABG) dementia, anesthesia induced memory loss, Huntington's disease and many other. It is apparent that a metabolic intervention may aid people suffering from such afflictions. Yet such therapies are not available at the present time.

Acetyl-coenzyme A carboxylase (ACC) (EC 6.4.1.2) catalyzes the carboxylation of acetyl-CoA to produce malonyl-CoA.

ATP+acetyl-CoA+HCO3-=ADP+phosphate+malonyl-CoA

By producing malonyl-CoA, ACC plays a key role in the regulation of fatty acid synthesis. Malonyl-CoA inhibits carnitine palmitoyltransferase I, thereby blocking the oxidation of fatty acids. In the absence of malonyl-CoA fatty acid oxidation can run unregulated and lead to the production of ketone bodies. In mammals, there are two isoforms of the ACC; ACC1 and ACC2. ACC1 is expressed in lipogenic tissues (e.g. liver, adipose, lactating mammary gland and others). In these tissues the malonyl-CoA produced by ACC is used in the biosynthesis of long-chain fatty acids by the fatty acid synthase complex (FAS). The long-chain fatty acids can then be incorporated into triacylglycerides and phospholipids. In contrast, ACC2 is expressed mostly in the heart and skeletal muscle, and in these tissues malonyl-CoA functions mainly as a potent inhibitor of fatty acid oxidation (for review see (Tong 2005)).

Mice engineered to carry a knockout of the muscle form of ACC, ACC2, have revealed that inhibition of ACC has numerous metabolic outcomes. Due to the lack of ACC activity, these animals have drastically reduced levels of malonyl-CoA in their heart and skeletal muscle. As a result of the depletion of malonyl-CoA, ACC2−/− mice exhibit continuous fatty acid oxidation, reduced body fat mass and body weight, despite consuming more food (hyperphagia). They are also protected against diabetes and obesity induced by high fat/high carbohydrate diets. These observations suggest that inhibitors of ACC2 may be novel therapeutic agents against obesity, diabetes and metabolic syndrome in general. In addition these mice exhibited lower insulin and glucose while at the same time elevated ketone body levels despite consuming a high carbohydrate/high fat diet (Abu-Elheiga, Oh et al. 2003).

The inventor has previously shown that induction of ketosis by oral administration of medium chain triglycerides (MCT) improves the cognitive performance of probable mild to moderate Alzheimer's disease subjects (U.S. Pat. No. 6,835,750; {Reger, 2004 #136}. However, this treatment requires large amounts of MCT to be administered and causes some intestinal distress. There is a need to solve problems associated with administration of MCT.

There remains a great need for treatment of disorders such as Age Associated Memory Impairment (AAMI), Mild Cognitive Impairment (MCI), Alzheimer's disease, Parkinson's disease, Friedreich's Ataxia (FRDA), GLUT1-deficient Epilepsy, Leprechaunism and Rabson-Mendenhall Syndrome, Coronary Arterial Bypass Graft (CABG) dementia, anesthesia induced memory loss, Huntington's disease and many others. For example, current treatments for Alzheimer's disease do little to slow or treat the disease.

### SUMMARY OF THE INVENTION

In one embodiment, the present invention includes a method of treating loss of cognitive function caused by reduced neuronal metabolism, wherein said treatment comprises administration of a pharmaceutical composition comprising a compound capable of inhibiting Acetyl CoA Carboxylase to a patient in need thereof in an amount sufficient to cause hyperketonemia in the patient, resulting in ketone bodies being utilized for energy in the brain. In one embodiment, the level of D-beta-hydroxybutyrate in the patient is raised to about 0.1 to 50 mM; is raised to about 0.2 to 20 mM; is raised to about 0.3 to 5 mM; is raised to about 0.5 to 2 mM; is raised to about 1 to 10 mM.

Another embodiment of the present invention includes selecting a patient for treatment with a compound capable of elevating ketone body concentrations, which includes the following steps, in no particular order; selecting a patient having a loss of cognitive function caused by reduced neuronal metabolism; determining a patient's apolipoprotein E genotype; and providing a pharmaceutical composition comprising an acetyl CoA carboxylase inhibitor to a patient having an absence of APOE4 in an amount effective for the treating loss of cognitive function caused by reduced neuronal metabolism.

In one embodiment of the present invention, the loss of cognitive function and/or reduced neuronal metabolism is caused by Age Associated Memory Impairment (AAMI), Mild Cognitive Impairment (MCI), Alzheimer's disease, Parkinson's disease, Friedreich's Ataxia (FRDA), GLUT1-deficient Epilepsy, Leprechaunism and Rabson-Mendenhall Syndrome, Coronary Arterial Bypass Graft (CABG) dementia, anesthesia induced memory loss, and/or Huntington's disease. In another embodiment of the present invention, the patient's apolipoprotein E genotype is APOE4 (−). The present invention also includes a pharmaceutical composition which causes hyperketonemia in the patient when the patient has a diet wherein carbohydrate intake is not restricted.

In another embodiment, a suitable acetyl CoA carboxylase inhibitor can be one or more of the following: [(3R)-1-[1-(anthracene-9-carbonyl)piperidin-4-yl]piperidin-3-yl]-morpholin-4-ylmethanone (also called (R)-anthracen-9-yl(3-(piperidine-1-carbonyl)-1,4′-bipiperidin-1′-yl)methanone herein) (CP 640186); CP-610432 (S-1′-(anthracene-9-carbonyl)-N,N-diethyl-1,4′-bipiperidine-3-carboxamide); CP-610431 (R-1′-(anthracene-9-carbonyl)-N,N-diethyl-1,4′-bipiperidine-3-carboxamide); CP-497485 (1′-(anthracene-9-carbonyl)-N,N-diethyl-1,4′-bipiperidine-3-carboxamide); phenylmethyl 5-(1-{[(2-{[N-(2,4-dihydroxy-3,3-dimethylbutanyl)-5-(6-aminooctahydro-9H-purin-9-yl)-4-(hydroxyl-2-[(phosphonooxy)tetrahydrofuran-2-yl]methyl dihydrogen diphosphate-β-alanyl]amino}ethyl)thio]acetyl}-2-oxohexahydro-1H-thieno[3,4-d]imidazol-4-yl)pentanoate; 5-(tetradecyloxy)-2-furan-carboxylic acid (TOFA; 3,3-dimethylhexanoate, monoglyceride (AC-0417-9); MEDICA 16 (β,β,β′,β′-tetramethylhexadecanoic acid); ESP-55016 (8-hydroxy-2,2, 14,14-tetra-methylpentadecanediotic acid); S2E ((+)-p-[1-p-tert-butylphenyl)-2-oxo-4-pyrrolidinyl]methoxybenzoic acid), and

1S,2S,3E,5R,6S,11S,14S,15R,16R,17S,18S)-15,17-dihydroxy-5,6,16-trimethoxy-2,14,18-trimethyl-11-phenyl-12,19-dioxabicyclo[13.3.1]nonadec-3-en-13-one (Soraphen A); 1′-N-Chloroacetamido-biotin, benzyl ester (CABI).

### DETAILED DESCRIPTION OF THE INVENTION

It is the novel insight of the present invention that inhibition of acetyl-CoA carboxylase will treat, prevent, inhibit or alleviate diseases associated with hypometabolism and/or loss of cognitive function caused by reduced neuronal metabolism, such as Age Associated Memory Impairment (AAMI), Mild Cognitive Impairment (MCI), Alzheimer's disease, Parkinson's disease, Friedreich's Ataxia (FRDA), GLUT1-deficient Epilepsy, Leprechaunism and Rabson-Mendenhall Syndrome, Coronary Arterial Bypass Graft (CABG) dementia, anesthesia induced memory loss, Huntington's disease and many others.

The present invention describes inducing hyperketonemia by administration to a mammal an inhibitor of the enzyme acetyl-CoA carboxylase and thereby increasing circulating ketone body levels. Ketone bodies, in particular β-hydroxybutyrate (βHB) and acetoacetate serve a critical role in the development and health of cerebral neurons. Numerous studies have shown that the preferred substrates for the developing mammalian neonatal brain are ketone bodies. There is a large body of evidence demonstrating that ketone bodies are used in a concentration dependent manner by the adult human brain, even in the elderly. The ability of ketone bodies to supplement glucose in the brain has been used to treat conditions of low glucose availability to the brain. Glut-1 is a constitutive glucose transporter that transports glucose into the central nervous system (CNS). The high glucose requirement of the brain requires that two functional copies of the GLUT-1 gene be present. If one copy of GLUT-1 is non-functional this results in GLUT-1 deficiency syndrome. The resulting low glucose levels during development lead to infant seizures, delayed development and microcephaly. Partial relief of these symptoms can be achieved by increasing serum ketone levels by administration of a ketogenic diet.

It is the novel insight of the inventor that induction of ketosis will alleviate conditions associated with decreased neuronal glucose utilization, and that inhibitors of acetyl CoA carboxylase will bring about ketosis, regardless of the metabolic state of the patient.

Incorporated by reference herein in their entireties are priority application U.S. Provisional Application No. 60/917,886, entitled “Inhibitors of Acetyl-CoA Carboxylase for Treatment of Hypometabolism,” and filed May 14, 2007; U.S. Pat. No. 6,835,750, entitled “Use of Medium Chain Triglycerides for the Treatment and Prevention of Alzheimer's Disease and Other Diseases Resulting from Reduced Neuronal Metabolism,” filed May 20, 2002; U.S. patent application Ser. No. 11/021,920, entitled “Use of Medium Chain Triglycerides for the Treatment and Prevention of Alzheimer's Disease and Other Diseases Resulting from Reduced Neuronal Metabolism II,” filed Dec. 22, 2004; U.S. patent application Ser. No. 11/331,673, entitled “Use of Medium Chain Triglycerides for the Treatment and Prevention of Alzheimer's Disease and Other Diseases Resulting from Reduced Neuronal Metabolism,” filed Jan. 13, 2006; U.S. patent application Ser. No. 11/611,114, entitled “Compositions and Methods for Improving or Preserving Brain Function,” filed Dec. 14, 2006; U.S. patent application Ser. No. 11/771,431 entitled “Combinations of Medium Chain Triglycerides and Therapeutic Agents for the Treatment and Prevention of Alzheimer's Disease and Other Diseases Resulting from Reduced Neuronal Metabolism,” filed Jun. 29, 2007; U.S. patent application Ser. No. 11/123,706, entitled “Method for Reducing Levels of Disease Associated Proteins,” filed May 3, 2005; U.S. patent application Ser. No. 11/424,429, entitled “Method To Reduce Oxidative Damage And Improve Mitochondrial Efficiency,” filed Jun. 15, 2006; and U.S. patent application Ser. No. 12/064,850, entitled “Use of Ketogenic Compounds for Treatment of Age-Associated Memory Impairment,” filed Feb. 26, 2008.

In one embodiment, the present invention includes a method of treating loss of cognitive function caused by reduced neuronal metabolism, wherein said treatment comprises administration of a pharmaceutical composition comprising a compound capable of inhibiting Acetyl CoA Carboxylase (ACC) to a patient in need thereof in an amount sufficient to cause hyperketonemia in the patient, resulting in ketone bodies being utilized for energy in the brain. Therapeutically effective amounts of the pharmaceutical compositions comprising compounds capable of inhibiting ACC can be any amount or dose sufficient to bring about the desired anti-dementia effect and depend, in part, on the severity and stage of the condition, the size and condition of the patient, as well as other factors readily known to those skilled in the art. The dosages can be given as a single dose, or as several doses, for example, divided over the course of several weeks.

In one embodiment, the pharmaceutical compositions are administered orally. In another embodiment, the pharmaceutical compositions are administered intravenously. Oral administration of pharmaceutical compositions and preparations of intravenous pharmaceutical compositions solutions are well known to those skilled in the art.

Oral and intravenous administration of pharmaceutical compositions of the present invention result in hyperketonemia. Hyperketonemia results in ketone bodies being utilized for energy in the brain even in the presence of glucose. Additionally, hyperketonemia results in a substantial (39%) increase in cerebral blood flow. Hyperketonemia has been reported to reduce cognitive dysfunction associated with systemic hypoglycemia in normal humans. Systemic hypoglycemia is distinct from the local defects in glucose metabolism that occur in AD.

This invention also provides a therapeutic agent for the treatment or prevention of loss of cognitive function caused by reduced neuronal metabolism, comprising pharmaceutical compositions comprising a compound capable of inhibiting Acetyl CoA Carboxylase (ACC) to a patient in need thereof in an amount sufficient to cause hyperketonemia in the patient, resulting in ketone bodies being utilized for energy in the brain. In a preferred embodiment, the therapeutic agent is provided in administratively convenient formulations of the compositions including dosage units incorporated into a variety of containers. Dosages of the therapeutic agent are preferably administered in an effective amount, in order to produce ketone body concentrations sufficient to increase the cognitive ability of patients afflicted loss of cognitive function caused by reduced neuronal metabolism. For example, blood ketone body levels are raised to about 0.1-50 mM, are raised to about 0.1-50 mM (either directly, or measured by urinary excretion in the range of about 5 mg/dL to about 160 mg/dL), raised to about 0.2-20 mM, raised to about 0.3-5 mM, raised to about 0.5-2 mM, although variations will necessarily occur depending on the formulation and host, for example. Ketosis induced by inhibition of Acetyl-CoA Carboxylase can be detected by several methods well known to those skilled in the art.

Different methods have been developed for monitoring of ketone bodies and include: urine dipsticks, laboratory readings, and capillary β-hydroxybutyrate (BHB) meters. Urine dipsticks measure only acetoacetate (ACA) and not BHB the most prevalent ketone during ketosis. Laboratory methods typically utilize enzymatic color reactions and can be used to measure levels of BHB, ACA or both, such tests are the most accurate. Capillary BHB meters can be used on whole blood and measure only BHB, but these meters are useful for measurements on blood drops and do not require a blood draw. Any of these methods can be used to determine ketosis as described in the present application. A typical ratio of BHB to ACA is about 3:1. Conversions of a level of BHB and total ketone body concentrations may be achieved by one of skill in the art.

Effective amount dosages of compounds capable of inhibiting ACC will be apparent to those skilled in the art.

Convenient unit dosage containers and/or formulations include tablets, capsules, lozenges, troches, hard candies, nutritional bars, nutritional drinks, metered sprays, creams, and suppositories, among others. The compositions may be combined with a pharmaceutically acceptable excipient such as gelatin, an oil, and/or other pharmaceutically active agent(s). For example, the compositions may be advantageously combined and/or used in combination with other therapeutic or prophylactic agents, different from the subject compounds. In many instances, administration in conjunction with the subject compositions enhances the efficacy of such agents. For example, the compounds may be advantageously used in conjunction with antioxidants, compounds that enhance the efficiency of glucose utilization, and mixtures thereof, (see e.g. Goodman et al. 1996).

Appropriate dosages with which to dose patients can be determined by one of skill in the art. In particular, guidance exists in the art for appropriate dosages to inhibit ACC in mammals for many if not all of the compounds referenced herein. Appropriate dosages of each compound taught herein that are efficacious for preventing or treating loss of cognitive function caused by reduced neuronal metabolism may be determined, for example, by dosing a mammal such as a mouse, at various levels and determining the dose which yields appropriate levels of D-beta-hydroxybutyrate in the blood after dosing. Such data can be extrapolated to humans. Appropriate levels of levels of D-beta-hydroxybutyrate in the blood can be, for example, about 0.1-50 mM (either directly, or measured by urinary excretion in the range of about 5 mg/dL to about 160 mg/dL), more preferably raised to about 0.2-20 mM, more preferably raised to about 0.3-5 mM, more preferably raised to about 0.5-2 mM. The inventors have found that in one embodiment, for example, D-beta-hydroxybutyrate blood levels of at least about 0.2 mM are efficacious to treat loss of cognitive function caused by reduced neuronal metabolism. In other embodiments, D-beta-hydroxybutyrate blood levels of at least about 0.1 mM, at least about 0.2 mM, at least about 0.25 mM, at least about 0.3 mM, at least about 0.35 mM, at least about 0.4 mM, at least about 0.45 mM, at least about 0.5 mM, at least about 0.55 mM, at least about 0.6 mM, at least about 0.7 mM, at least about 0.8 mM, at least about 0.9 mM, and/or at least about 1 mM are efficacious to treat loss of cognitive function caused by reduced neuronal metabolism

In some embodiments, the blood level of D-beta-hydroxybutyrate in the patient is raised to about 0.1 to 50 mM; raised to about 0.2 to 20 mM; raised to about 0.3 to 5 mM; raised to about 0.5 to 2 mM; or raised to about 1 to 10 mM.

The invention also provides pharmaceutical compositions comprising a therapeutically effective amount of a compound as described herein in a combination with a pharmaceutically acceptable carrier. The compositions comprise compounds of the invention formulated together with one or more non-toxic pharmaceutically acceptable carriers. The pharmaceutical compositions can be formulated for oral administration in solid or liquid form, for parenteral injection or for rectal administration

The term “therapeutically acceptable carrier” as used herein, means a non-toxic, solid, semi-solid or liquid filler, diluent, encapsulating material, or formulation auxiliary of any type. Examples of therapeutically suitable excipients include sugars; cellulose and derivatives thereof; oils; glycols; solutions; buffering, coloring, releasing, coating, sweetening, flavoring, and perfuming agents; and the like. These therapeutic compositions can be administered parenterally, intracisternally, orally, rectally, or intraperitoneally.

Liquid dosage forms for oral administration of the present compounds comprise formulations of the same as emulsions, microemulsions, solutions, suspensions, syrups, and elixirs. In addition to the compounds, the liquid dosage forms can contain diluents and/or solubilizing or emulsifying agents. Besides inert diluents, the oral compositions can include wetting, emulsifying, sweetening, flavoring, and perfuming agents.

Injectable preparations of the present compounds comprise sterile, injectable, aqueous and oleaginous solutions, suspensions or emulsions, any of which can be optionally formulated with parenterally suitable diluents, dispersing, wetting, or suspending agents. These injectable preparations can be sterilized by filtration through a bacterial-retaining filter or formulated with sterilizing agents that dissolve or disperse in the injectable media.

Inhibition of ACC by the compounds of the present invention can be delayed by using a liquid suspension of crystalline or amorphous material with poor water solubility. The rate of absorption of the compounds depends upon their rate of dissolution, which, in turn, depends on their crystallinity. Delayed absorption of a parenterally administered compound can be accomplished by dissolving or suspending the compound in oil. Injectable depot forms of the compounds can also be prepared by microencapsulating the same in biodegradable polymers. Depending upon the ratio of compound to polymer and the nature of the polymer employed, the rate of release can be controlled. Depot injectable formulations are also prepared by entrapping the compounds in liposomes or microemulsions that are compatible with body tissues.

Solid dosage forms for oral administration of the present compounds include capsules, tablets, pills, powders, and granules In such forms, the compound is mixed with at least one inert, therapeutically suitable excipient such as a carrier, filler, extender, disintegrating agent, solution retarding agent, wetting agent, absorbent, or lubricant. With capsules, tablets, and pills, the excipient can also contain buffering agents. Suppositories for rectal administration can be prepared by mixing the compounds with a suitable non-irritating excipient that is solid at ordinary temperature but fluid in the rectum.

The present compounds can be microencapsulating with one or more of the excipients discussed previously The solid dosage forms of tablets, dragees, capsules, pills, and granules can be prepared with coatings and shells such as enteric and release controlling In these forms, the compounds can be mixed with at least one inert diluent and can optionally comprise tableting lubricants and aids. Capsules can also optionally contain opacifying agents that delay release of the compounds in a desired part of the intestinal tract.

Transdermal patches have the added advantage of providing controlled delivery of the present compounds to the body. Such dosage forms are prepared by dissolving or dispensing the compounds in the proper medium. Absorption enhancers can also be used to increase the flux of the compounds across the skin, and the rate of absorption can be controlled by providing a rate controlling membrane or by dispersing the compounds in a polymer matrix or gel.

The compounds of the invention can be used in the form of pharmaceutically acceptable salts, esters, or amides derived from inorganic or organic acids. The term “pharmaceutically acceptable salts, esters and amides,” as used herein, include salts, zwitterions, esters and amides of compounds of formula (I) which are, within the scope of sound medical judgment, suitable for use in contact with the tissues of humans and lower animals without undue toxicity, irritation, allergic response, and the like, are commensurate with a reasonable benefit/risk ratio, and are effective for their intended use,

Pharmaceutically acceptable salts are well known in the art. The salts can be prepared during the final isolation and purification of the compounds or separately by reacting an amino group of the compounds with a suitable acid. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetic, trifluoroacetic, glutamate, para-toluenesulfonate, undecanoate, hydrochloric, hydrobromic, sulfuric, phosphoric, and the like. The amino groups of the compounds can also be quaternized with alkyl chlorides, bromides, and iodides such as methyl, ethyl, propyl, isopropyl, butyl, lauryl, myristyl, stearyl, and the like. The present invention contemplates pharmaceutically suitable salts formed at the nitrogen of formula (I).

Disorders that can be treated or prevented in a patient by administering to the patient, a therapeutically effective amount of compound of the present invention in such an amount and for such time as is necessary to achieve the desired result. The term “therapeutically effective amount,” refers to a sufficient amount of a compound of formula (I) to effectively ameliorate disorders by inhibiting ACC at a reasonable benefit/risk ratio applicable to any medical treatment. The specific therapeutically effective dose level for any particular patient depends upon a variety of factors including the disorder being treated and the severity of the disorder; the activity of the compound employed; the specific composition employed; the age, body weight, general health, sex, and diet of the patient; the time of administration, route of administration, rate of excretion; the duration of the treatment; and drugs used in combination or coincidental therapy.

The total daily dose of the compounds of the present invention necessary to inhibit the action of ACC in single or divided doses can be in amounts, for example, from about 0.1 to 50 mg/kg body weight. In a more preferred range, compounds of the present invention inhibit the action of ACC in a single or divided doses from about 1 to 25 mg/kg body weight, Single dose compositions can contain such amounts or submultiple doses thereof of the compounds of the present invention to make up the daily dose. In general, treatment regimens comprise administration to a patient in need of such treatment from about 1 mg to about 1000 mg of the compounds per day in single or multiple doses.

In one embodiment, the compounds capable of inhibiting ACC are effective to treat and/or prevent loss of cognitive function caused by reduced neuronal metabolism. In some embodiments, the loss of cognitive function and/or reduced neuronal metabolism is caused by Alzheimer's disease or Mild Cognitive Impairment. In other embodiments, loss of cognitive function and/or reduced neuronal metabolism is caused by reduced neuronal metabolism in diseases such as Age Associated Memory Impairment (AAMI), Mild Cognitive Impairment (MCI), Alzheimer's disease, Parkinson's disease, Friedreich's Ataxia (FRDA), GLUT1-deficient Epilepsy, Leprechaunism and Rabson-Mendenhall Syndrome, Coronary Arterial Bypass Graft (CABG) dementia, anesthesia induced memory loss, and/or Huntington's disease.

In one embodiment, use of any of the pharmaceutical compositions of the present invention causes hyperketonemia in the patient when the patient has a diet wherein carbohydrate intake is not restricted. Hyperketonemia results in ketone bodies being utilized for energy in the brain even in the presence of glucose. Additionally, hyperketonemia results in a substantial (39%) increase in cerebral blood flow.

Although suitable ACC inhibitors are discussed more fully elsewhere herein, exemplary acetyl CoA carboxylase inhibitors can comprise one or more of any of the following compounds: [(3R)-1-[1-(anthracene-9-carbonyl)piperidin-4-yl]piperidin-3-yl]-morpholin-4-ylmethanone (also called (R)-anthracen-9-yl(3-(piperidine-1-carbonyl)-1,4′-bipiperidin-1′-yl)methanone herein) (CP 640186); CP-610432 (S-1′-(anthracene-9-carbonyl)-N,N-diethyl-1,4′-bipiperidine-3-carboxamide); CP-610431 (R-1′-(anthracene-9-carbonyl)-N,N-diethyl-1,4′-bipiperidine-3-carboxamide); CP-497485 (1′-(anthracene-9-carbonyl)-N,N-diethyl-1,4′-bipiperidine-3-carboxamide); phenylmethyl 5-(1-{[(2-{[N-(2,4-dihydroxy-3,3-dimethylbutanyl)-5-(6-aminooctahydro-9H-purin-9-yl)-4-(hydroxyl-2-[(phosphonooxy)tetrahydrofuran-2-yl]methyl dihydrogen diphosphate-β-alanyl]amino}ethyl)thio]acetyl}-2-oxohexahydro-1H-thieno[3,4-d]imidazol-4-yl)pentanoate; 5-(tetradecyloxy)-2-furan-carboxylic acid (TOFA); 3,3-dimethylhexanoate, monoglyceride (AC-0417-9); MEDICA 16 (β,β,β′,β′-tetramethylhexadecanoic acid); ESP-55016 (8-hydroxy-2,2,14,14-tetra-methylpentadecanediotic acid); S2E ((+)-p-[1-p-tert-butylphenyl)-2-oxo-4-pyrrolidinyl]methoxybenzoic acid); and 1S,2S,3E,5R,6S,11S,14S,15R,16R,17S,18S)-15,17-dihydroxy-5,6,16-trimethoxy-2,14,18-trimethyl-11-phenyl-12,19-dioxabicyclo[13.3.1]nonadec-3-en-13-one (Soraphen A)); 1′-N-Chloroacetamido-biotin, benzyl ester (CABI).

Appropriate dosages for each of these exemplary compounds can be determined by those of skill in the art. For [(3R)-1-[1-(anthracene-9-carbonyl)piperidin-4-yl]piperidin-3-yl]-morpholin-4-ylmethanone (also called (R)-anthracen-9-yl(3-(piperidine-1-carbonyl)-1,4′-bipiperidin-1′-yl)methanone herein) (CP 640186) and related compounds CP-497485, CP-610431, and CP-610432, these compounds are specific inhibitors of ACC and are efficacious to inhibit ACC at a fairly low dosing. In one embodiment, these inhibitors are dosed at between about 1 mg/kg and about 10 mg/kg; in another embodiment, these inhibitors are dosed at between about 5 mg/kg and 10 mg/kg. Precise dosages to stimulate desired ketone body levels in the blood can be easily determined by those with skill.

For phenylmethyl 5-(1-{[(2-{[N-(2,4-dihydroxy-3,3-dimethylbutanyl)-5-(6-aminooctahydro-9H-purin-9-yl)-4-(hydroxyl-2-[(phosphonooxy)tetrahydrofuran-2-yl]methyl dihydrogen diphosphate-β-alanyl]amino}ethyl)thio]acetyl}-2-oxohexahydro-1H-thieno[3,4-d]imidazol-4-yl)pentanoate and related compounds, these compounds are CoA sequestration agents and are efficacious to inhibit ACC at a wide range of dosing. In one embodiment, these inhibitors are dosed at between about 500 mg/kg and about 2000 mg/kg; in another embodiment, these inhibitors are dosed at between about 1000 mg/kg and 2000 mg/kg. Precise dosages to stimulate desired ketone body levels in the blood can be easily determined by those with skill.

For 5-(tetradecyloxy)-2-furan-carboxylic acid (TOFA) and related compounds, these compounds are CoA sequestration agents and are efficacious to inhibit ACC at a wide range of dosing. In one embodiment, these inhibitors are dosed at between about 0.5 mg/kg and about 1000 mg/kg; in another, between about 0.5 mg/kg and about 5 mg/kg; in another, about 200 mg/kg and about 1000 mg/kg. Precise dosages to stimulate desired ketone body levels in the blood can be easily determined by those with skill.

For 3,3-dimethylhexanoate, monoglyceride (AC-0417-9) and related compounds, these compounds are CoA sequestration agents and are efficacious to inhibit ACC at a wide range of dosing. In one embodiment, these inhibitors are dosed at between about 500 mg/kg and about 3000 mg/kg; in another, between about 1000 mg/kg and about 2000 mg/kg; in another, about 1500 mg/kg. Precise dosages to stimulate desired ketone body levels in the blood can be easily determined by those with skill.

For MEDICA 16 (β,β,β′,β′-tetramethylhexadecanoic acid) and related compounds, these compounds are CoA sequestration agents and are efficacious to inhibit ACC at a wide range of dosing. In one embodiment, these inhibitors are dosed at between about 500 mg/kg and about 3000 mg/kg; in another, between about 1000 mg/kg and about 2000 mg/kg; in another, about 15000 mg/kg. Precise dosages to stimulate desired ketone body levels in the blood can be easily determined by those with skill.

For ESP-55016 (8-hydroxy-2,2,14,14-tetra-methylpentadecanediotic acid) and related compounds, these compounds are CoA sequestration agents and are efficacious to inhibit ACC at a wide range of dosing. In one embodiment, these inhibitors are dosed at between about 500 mg/kg and about 3000 mg/kg; in another, between about 1000 mg/kg and about 2000 mg/kg; in another, about 15000 mg/kg. Precise dosages to stimulate desired ketone body levels in the blood can be easily determined by those with skill.

For S2E ((+)-p-[1-p-tert-butylphenyl)-2-oxo-4-pyrrolidinyl]methoxybenzoic acid) and related compounds, these compounds are CoA sequestration agents and are efficacious to inhibit ACC at a wide range of dosing. In one embodiment, these inhibitors are dosed at between about 500 mg/kg and about 3000 mg/kg; in another, between about 1000 mg/kg and about 2000 mg/kg; in another, about 15000 mg/kg. Precise dosages to stimulate desired ketone body levels in the blood can be easily determined by those with skill.

For 1S,2S,3E,5R,6S,11S,14S,15R,16R,17S,18S)-15,17-dihydroxy-5,6,16-trimethoxy-2,14,18-trimethyl-11-phenyl-12,19-dioxabicyclo[13.3.1]nonadec-3-en-13-one (Soraphen A) and related compounds, these compounds are specific inhibitors of ACC and are efficacious to inhibit ACC at a low range of dosing. In one embodiment, these inhibitors are dosed at between about 1 mg/kg and about 10 mg/kg; in another embodiment, these inhibitors are dosed at between about 5 mg/kg and 10 mg/kg. Precise dosages to stimulate desired ketone body levels in the blood can be easily determined by those with skill.

In another embodiment, the invention provides the subject compounds in the form of one or more prodrugs, which can be metabolically converted to the subject compounds by the recipient host. As used herein, a prodrug is a compound that exhibits pharmacological activity after undergoing a chemical transformation in the body. The said prodrugs will be administered in a dosage required to increase blood ketone bodies to a level which is useful to treat loss of cognitive function caused by reduced neuronal metabolism. A wide variety of prodrug formulations are known in the art. For example, prodrug bonds may be hydrolyzable, such as esters or anhydrides, or enzymatically biodegradable, such as amides.

In one embodiment, the invention can comprise a pharmaceutical composition, and/or provides a pharmaceutical composition, comprising a mixture of an ACC inhibitor and another compound capable of raising ketone body levels, such as, for example, medium chain triglyceride (MCT). The nature of such pharmaceutical compositions will depend on the duration and route of administration. Such formulations will be in the range of about 0.05 g/kg/day to 10 g/kg/day of MCT and about 0.05 mg/kg/day to 10 mg/kg/day of carnitine or its derivatives. In one embodiment, an MCT dose will be in the range of about 0.05 g/kg/day to 10 g/kg/day of MCT. In another embodiment, the dose will be in the range of about 0.25 g/kg/day to 5 g/kg/day of MCT. In another embodiment, the dose will be in the range of about 0.5 g/kg/day to 2 g/kg/day of MCT. In another embodiment, the dose will be in the range of about 0.25 g/kg/day to about 0.5 g/kg/day. Such amounts may readily be converted to g/day using the standard 68 kg person. Variations will necessarily occur depending on the formulation and/or host, for example.

In one embodiment, the pharmaceutical compositions of the invention induce hyperketonemia for 3-4 hours in patients and/or human subjects. In another embodiment, the composition induces hyperketonemia in patients and/or human subjects for less than one hour; between 1-2 hours; between 2-3 hours; between 4-5 hours; between 5-6 hours; between 7-8 hours; between 9-10 hours; and/or ten hours or longer.

In another embodiment, the invention includes a pharmaceutical composition with an agent which enhances endogenous fatty acid metabolism by the recipient. The said therapeutic agent will be administered in a dosage required to increase or augment blood ketone bodies to a level required to treat and prevent the occurrence of Alzheimer's Disease. Ketone bodies are produced continuously by oxidation of fatty acids in tissues that are capable of such oxidation. The major organ for fatty acid oxidation is the liver. Under normal physiological conditions ketone bodies are rapidly utilized and cleared from the blood. Under some conditions, such as starvation or low carbohydrate diet, ketone bodies are produced in excess and accumulate in the blood stream. Compounds that mimic the effect of increasing oxidation of fatty acids will raise ketone body concentration to a level to provide an alternative energy source for neuronal cells with compromised metabolism. Since the efficacy of such compounds derives from their ability to increase fatty acid utilization and raise blood ketone body concentration they are dependent on the embodiments of the present invention. Compounds that mimic the effect of increasing oxidation of fatty acids and will raise ketone body concentration include but are not limited to the ketone bodies, D-β-hydroxybutyrate and aceotoacetate, and metabolic precursors of these. The term metabolic precursor, as used herein, refers to compounds that comprise 1,3 butane diol, acetoacetyl or D-β-hydroxybutyrate moieties such as acetoacetyl-1-1,3-butane diol, acetoacetyl-D-β-hydroxybutyate, and acetoacetylglycerol. Esters of any such compounds with monohydric, dihydric or trihydric alcohols is also envisaged. Metabolic precursors also include polyesters of D-β-hydroxybutyrate, and acetoaoacetate esters of D-β-hydroxybutyrate. Polyesters of D-β-hydroxybutyrate include oligomers of this polymer designed to be readily digestible and/or metabolized by humans or animals. These preferably are of 2 to 100 repeats long, typically 2 to 20 repeats long, and most conveniently from 3 to 10 repeats long. Examples of poly D-β-hydroxybutyrate or terminally oxidized poly-D-β-hydroxybutyrate esters useable as ketone body precursors are given below:

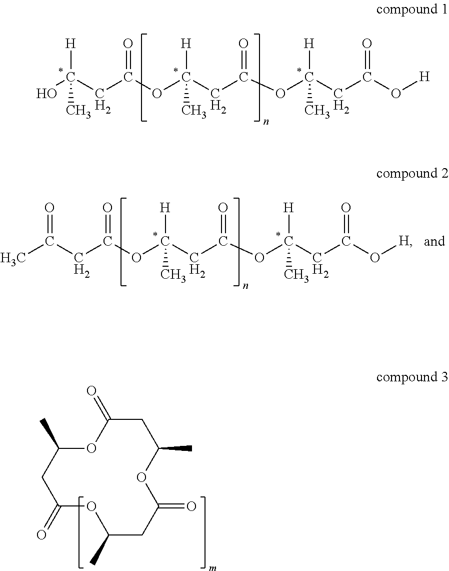

In each case n is selected such that the polymer or oligomer is readily metabolized on administration to a human or animal body to provide elevated ketone body levels in blood. Preferred values of n are integers of 0 to 1,000, more preferably 0 to 200, still more preferably 1 to 50, most preferably 1 to 20, particularly conveniently being from 3 to 5. In each case m is an integer of 1 or more, a complex thereof with one or more cations or a salt thereof for use in therapy or nutrition. Examples of cations and typical physiological salts are described herein, and additionally include sodium, potassium, magnesium, calcium, each balanced by a physiological counter-ion forming a salt complex, L-lysine, L-arginine, methyl glucamine, and others known to those skilled in the art. The preparation and use of such metabolic precursors is detailed in Veech, WO 98/41201, and Veech, WO 00/15216, each of which is incorporated by reference herein in its entirety.

In another embodiment, the invention provides a therapeutic compound or mixture of compounds, the composition and dosage of which is influenced by the patients' genotype, in particular the alleles of apoliproprotein E gene. Elswhere the inventor discloses that non-E4 carriers performed better than those with the E4 allele when elevated ketone body levels were induced. Also, those with the E4 allele had higher fasting ketone body levels and the levels continued to rise at the two hour time interval. Therefore, E4 carriers may require higher ketone levels or agents that increase the ability to use the ketone bodies that are present.

Accordingly, in one embodiment, the invention includes a method of selecting a patient for treatment with a compound capable of elevating ketone body concentrations, comprising: a) selecting a patient having loss of cognitive function caused by reduced neuronal metabolism; b) determining a patient's apolipoprotein E genotype; and

c) providing a pharmaceutical composition comprising an acetyl CoA carboxylase inhibitor to a patient having an absence of APOE4 in an amount effective for the treating loss of cognitive function caused by reduced neuronal metabolism.

The methods of treating and or preventing loss of cognitive function caused by reduced neuronal metabolism also comprise administering a pharmaceutical composition of the present invention to a patient whose apolipoprotein E genotype comprises APOE4 (−).

Although suitable ACC inhibitors can comprise any ACC inhibitor known in the art, with specificity for any ACC inhibitor, such as, for example, plant ACC, prokaryotic ACC, or eukaryotic ACC. Described herein are a number of exemplary ACC inhibitors that are known in the rt. Each of these compounds and produgs of these compounds may be used with the present invention. Exemplary acetyl CoA carboxylase inhibitors can comprise one or more of any of the following compounds (a brief description of each of these compounds follows):

In one embodiment, a suitable ACC inhibitor is one or more of a class of compounds for treating or preventing loss of cognitive function caused by reduced neuronal metabolism are compounds as described in Formula 1. Formula I compounds are effective for the elevation of circulating ketone bodies and treatment, prevention, inhibition or alleviation of diseases associated with neuronal hypometabolism. Formula I is described in U.S. Pat. No. 6,979,741. Description of genus Formula I and all species described in U.S. Pat. No. 6,979,741, as well as all methods of production of the same, is specifically incorporated by reference herein. Additionally, the entire disclosure of U.S. Pat. No. 6,979,741 is incorporated herein by reference in its entirety. Briefly, a compound of Formula I is described as

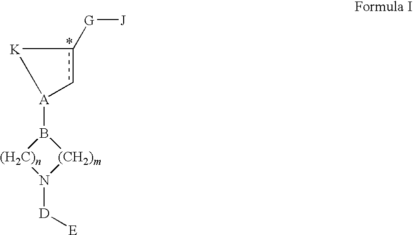

prodrugs thereof, or pharmaceutically acceptable salts of said compounds or of said prodrugs;
wherein A-B is N—CH or CH—N;
K is (CH₂)ᵣ wherein r is 2, 3 or 4;
m and n are each independently 1, 2 or 3 when A-B is N—CH or m and n are each independently 2 or 3 when A-B is CH—N;
the dashed line represents the presence of an optional double bond;
D is carbonyl or sulfonyl;
E is either
a.) a bicyclic ring consisting of two fused fully unsaturated five to seven membered rings, taken independently, each of said rings optionally having one to four heteroatoms selected independently from oxygen, sulfur and nitrogen, or
b.) a tricyclic ring consisting of two fused fully unsaturated five to seven membered rings, taken independently, each of said rings optionally having one to four heteroatoms selected independently from oxygen, sulfur and nitrogen, said two fused rings fused to a third partially saturated, fully unsaturated or fully saturated five to seven membered ring, said third ring optionally having one to four heteroatoms selected independently from oxygen, sulfur and nitrogen; or
c.) a tetracyclic ring comprising a bicyclic ring consisting of two fused fully unsaturated five to seven membered rings, taken independently, each of said rings optionally having one to four heteroatoms selected independently from oxygen, sulfur and nitrogen, said bicyclic ring fused to two fully saturated, partially saturated or fully unsaturated five to seven membered monocyclic rings taken independently, each of said rings optionally having one to four heteroatoms selected independently from oxygen, sulfur and nitrogen or said bicyclic ring fused to a second bicyclic ring consisting of two fused fully saturated, partially saturated or fully unsaturated five to seven membered rings, taken independently, each of said rings optionally having one to four heteroatoms selected independently from oxygen, sulfur and nitrogen; or
d.) a teraryl ring comprising a fully unsaturated five to seven membered ring, said ring optionally having one to four heteroatoms selected independently from oxygen, sulfur and nitrogen, and said ring di-substituted independently with a fully unsaturated five to seven membered ring to form a teraryl nonfused ring system, each of said substituent rings optionally having one to four heteroatoms selected independently from oxygen, sulfur and nitrogen,
wherein said E bi-, tri- or tetra cyclic ring or teraryl ring is optionally mono-, di- or tri-substituted independently on each ring used to form the bi-, tri- or tetra cyclic ring or teraryl ring with halo, hydroxy, amino, cyano, nitro, oxo, carboxy, (C₁-C₆)alkyl, (C₂-C₆)alkenyl, (C₂-C₆)alkynyl, (C₁-C₆)alkoxy, (C₁-C₄)alkylthio, (C₁-C₆)alkoxycarbonyl, (C₁-C₆) alkylcarbonyl, (C₁-C₆)alkylcarbonylamino, or mono-N— or di-N,N—(C₁-C₆)alkylamino, mono-N— or di-N,N—(C₁-C₆)alkylaminocarbonyl wherein said (C₁-C₆)alkyl, (C₁-C₆)alkoxy and (C₁-C₄)alkylthio substituents are also optionally mono-, di- or tri-substituted independently with chloro, bromo, hydroxy, (C₁-C₆)alkoxy, amino, mono-N— or di-N,N—(C₁-C₆)alkylamino or from one to nine fluorines; and
wherein said E bi-, tri- or tetra-cyclic ring or teraryl ring is optionally mono-substituted with a partially saturated, fully saturated or fully unsaturated three to eight membered ring R¹0 optionally having one to four heteroatoms selected independently from oxygen, sulfur and nitrogen or a bicyclic ring R¹1 consisting of two fused partially saturated, fully saturated or fully unsaturated three to eight membered rings, taken independently, each of said rings optionally having one to four heteroatoms selected independently from oxygen, sulfur and nitrogen, said R¹0 and R¹1 rings optionally additionally bridged and said R¹0 and R¹1 rings optionally linked through a fully saturated, partially unsaturated or fully unsaturated one to four membered straight or branched carbon chain wherein the carbon(s) may optionally be replaced with one or two heteroatoms selected independently from oxygen, nitrogen and sulfur, provided said E bicyclic ring has at least one substituent and the E bicyclic ring atom bonded to D is carbon;
wherein said R¹0 or R¹1 ring is optionally mono-, di- or tri-substituted independently with halo, hydroxy, amino, cyano, nitro, oxo, carboxy, (C₁-C₆)alkyl, (C₂-C₆)alkenyl, (C₂-C₆)alkynyl, (C₁-C₆)alkoxy, (C₁-C₄)alkylthio, (C₁-C₆)alkoxycarbonyl, (C₁-C₆) alkylcarbonyl, (C₁-C₆)alkylcarbonylamino, or mono-N— or di-N,N—(C₁-C₆)alkylamino or mono-N— or di-N,N—(C₁-C₆)alkylaminocarbonyl wherein said (C₁-C₆)alkyl and (C₁-C₆)alkoxy substituents are also optionally mono-, di- or tri-substituted independently with halo, hydroxy, (C₁-C₆)alkoxy, amino, mono-N— or di-N,N—(C₁-C₆)alkylamino or from one to nine fluorines;
G is carbonyl, sulfonyl or CR⁷, R⁸;
wherein R⁷ and R⁸ are each independently H, (C₁-C₆)alkyl, (C₂-C₆)alkenyl or (C₂-C₆)alkynyl or a five to seven membered partially saturated, fully saturated or fully unsaturated ring optionally having one heteroatom selected from oxygen, sulfur and nitrogen;

### J is OR¹, NR²R³ or CR⁴R⁵, R⁶;

wherein R¹, R² and R³ are each independently H, Q, or a (C₁-C₁0)alkyl, (C₃-C₁0)alkenyl or (C₃-C₁0)alkynyl substituent wherein said carbon(s) may optionally be replaced with one or two heteroatoms selected independently from oxygen, nitrogen and sulfur and wherein said sulfur is optionally mono- or di-substituted with oxo, said carbon(s) is optionally mono-substituted with oxo, said nitrogen is optionally di-substituted with oxo, said carbon(s) is optionally mono-, di- or tri-substituted independently with halo, hydroxy, amino, nitro, cyano, carboxy, (C₁-C₄)alkylthio, (C₁-C₆)alkyloxycarbonyl, mono-N— or di-N,N—(C₁-C₆)alkylamino or mono-N— or di-N,N—(C₁-C₆)alkylaminocarbonyl;
and said chain is optionally mono-substituted with Q¹;
wherein Q and Q¹ are each independently a partially saturated, fully saturated or fully unsaturated three to eight membered ring optionally having one to three heteroatoms selected independently from oxygen, sulfur and nitrogen or a bicyclic ring consisting of two fused or spirocyclic partially saturated, fully saturated or fully unsaturated three to six membered rings, taken independently, said bicyclic ring optionally having one to three heteroatoms selected independently from oxygen, sulfur and nitrogen, said mono or bicyclic ring optionally additionally bridged with (C₁-C₃)alkylene wherein said (C₁-C₃)alkylene carbons are optionally replaced with one to two heteroatoms selected independently from oxygen, sulfur and nitrogen;
wherein said Q and Q¹ ring are each independently optionally mono-, di-, tri-, or tetra-substituted independently with halo, hydroxy, amino, nitro, cyano, oxo, carboxy, (C₁-C₆) alkyl, (C₂-C₆)alkenyl, (C₂-C₆)alkynyl, (C₁-C₆)alkoxy, (C₁-C₄)alkylthio, (C₁-C₆) alkylcarbonyl, (C₁-C₆)alkylcarbonylamino, (C₁-C₆)alkyloxycarbonyl, mono-N— or di-N,N—(C₁-C₆)alkylamino, mono-N— or di-N,N—(C₁-C₆)alkylaminosulfonyl, mono-N— or di-N,N—(C₁-C₆)alkylaminocarbonyl, wherein said (C₁-C₆)alkyl substituent is optionally mono-, di- or tri-substituted independently with halo, hydroxy, amino, nitro, cyano, oxo, carboxy, (C₁-C₆)alkoxy, (C₁-C₄)alkylthio, (C₁-C₆)alkyloxycarbonyl or mono-N— or di-N,N—(C₁-C₆)alkylamino wherein said (C₁-C₆)alkyl substituent is also optionally substituted with from one to nine fluorines; or wherein R² and R³ can be taken together with the nitrogen atom to which they are attached to form a partially saturated, fully saturated or fully unsaturated three to eight membered ring optionally having one to three additional heteroatoms selected independently from oxygen, sulfur and nitrogen or a bicyclic ring consisting of two fused, bridged or spirocyclic partially saturated, fully saturated or fully unsaturated three to six membered rings, taken independently, said bicyclic ring optionally having one to three additional heteroatoms selected independently from oxygen, sulfur and nitrogen or a tricyclic ring consisting of three fused, bridged or spirocyclic partially saturated, fully saturated or fully unsaturated three to six membered rings, taken independently, said tricyclic ring optionally having one to three additional heteroatoms selected independently from oxygen, sulfur and nitrogen;
wherein said NR², R³ ring is optionally mono-, di-, tri- or tetra-substituted independently with R¹5, halo, hydroxy, amino, nitro, cyano, oxo, carboxy, (C₁-C₆) alkyl, (C₂-C₆)alkenyl, (C₂-C₆)alkynyl, (C₁-C₆)alkoxy, (C₁-C₄)alkylthio, (C₁-C₆)alkylcarbonylamino or mono-N— or di-N,N—(C₁-C₆)alkylamino, wherein said (C₁-C₆)alkyl substituent is optionally mono-, di- or tri-substituted independently with halo, hydroxy, amino, nitro, cyano, oxo, carboxy, (C₁-C₆)alkoxy, (C₁-C₄)alkylthio, (C₁-C₆)alkyloxycarbonyl, mono-N— or di-N,N—(C₁-C₆)alkylamino, said (C₁-C₆)alkyl substituent is also optionally substituted with from one to nine fluorines;
wherein R¹5 is carbonyl, carbamoyl, sulfonyl or sulfamoyl substituted with H, (C₁-C₆)alkyl, (C₁-C₆)alkyloxy, (C₁-C₆)alkyloxycarbonyl, (C₁-C₆)alkyloxycarbonyl(C₁-C₆)alkyl, mono-N— or di-N,N—(C₁-C₆)alkylamino, wherein said (C₁-C₆)alkyl substituent is optionally mono-, di- or tri-substituted independently with halo, hydroxy, amino, nitro, cyano, oxo, carboxy, (C₁-C₆)alkoxy, (C₁-C₄)alkylthio, (C₁-C₆)alkyloxycarbonyl, (C₁-C₆)alkylcarbonyloxy, mono-N— or di-N,N—(C₁-C₆)alkylamino or the R¹5 carbonyl, carbamoyl, sulfonyl or sulfamoyl linked substituent is a partially saturated, fully saturated or fully unsaturated three to eight membered ring optionally linked through (C₁-C₆)alkyl and optionally having one to three heteroatoms selected independently from oxygen, sulfur and nitrogen wherein said ring is optionally mono-, di- or tri-substituted with halo, hydroxy, amino, nitro, cyano, oxo, carboxy, (C₁-C₆) alkyl, (C₂-C₆)alkenyl, (C₂-C₆)alkynyl, (C₁-C₄)alkylthio, (C₁-C₆)alkoxy, (C₁-C₆)alkylcarbonylamino, mono-N— or di-N,N—(C₁-C₆)alkylamino;
wherein said NR², R³ ring is optionally substituted with a partially saturated, fully saturated or fully unsaturated three to eight membered ring optionally having one to three heteroatoms selected independently from oxygen, sulfur and nitrogen or a bicyclic ring consisting of two fused partially saturated, fully saturated or fully unsaturated three to six membered rings, taken independently, said bicyclic ring optionally having one to three heteroatoms selected independently from oxygen, sulfur and nitrogen, said mono or bicyclic ring optionally additionally bridged said ring optionally having one to three heteroatoms selected independently from oxygen, sulfur and nitrogen, wherein said (C₁-C₆)alkyl and said ring are optionally mono-, di- or tri-substituted with halo, hydroxy, amino, nitro, cyano, oxo, carboxy, (C₂-C₆)alkenyl, (C₃-C₆)alkynyl, (C₁-Cₛ)alkylcarbonylamino, hydroxy, (C₁-C₆)alkoxy, (C₁-C₄)alkylthio, (C₁-C₆)alkoxy, mono-N— or di-N,N—(C₁-C₆)alkylamino;
wherein R⁴, R⁵ and R⁶ are independently H, halo, hydroxy, (C₁-C₆)alkyl or R⁴ and R⁵ are taken together to form a partially saturated, fully saturated or fully unsaturated three to eight membered ring, said ring optionally having one to three heteroatoms selected independently from oxygen, sulfur and nitrogen, wherein said (C₁-C₆)alkyl and said ring are optionally mono-, di- or tri-substituted with halo, hydroxy, amino, nitro, cyano, oxo, carboxy, (C₂-C₆)alkenyl, (C₃-C₆)alkynyl, (C₁-C₆)alkylcarbonylamino, hydroxy, (C₁-C₆)alkoxy, (C₁-C₄)alkylthio, (C₁-C₆)alkoxy, mono-N— or di-N,N—(C₁-C₆)alkylamino
with the proviso that 1′-(anthracene-9-carbonyl)-[1,4′]bipiperidinyl-3-carboxylic acid diethylamide; 1′-(1-oxa-2,3-diaza-cyclopenta[a]naphthalene-5-sulfonyl)-[1,4′]bipiperidin yl-3 carboxylic acid diethylamide; 1′-(5-dimethylamino-naphthalene-1-sulfonyl)-[1,4′]bipiperidinyl-3-carboxylic acid diethylamide; 1′(9,10,10-trioxo-9,10-dihydro-thioxanthene-3-carbonyl)-[1-4′]bipiperidiny 1-3-carboxylic acid diethylamide; and 1′-(2-Oxo-2H-chromen-3-carbonyl)-[1,4′]bipiperidinyl-3-carboxylic acid diethylamide are not included.

Included within this genus, in addition to all species described in U.S. Pat. No. 6,979,741, are substituted bipiperidylcarboxamides. A preferred group of compounds conforming to Formula I are the compounds (3R)-Anthracen-9-yl-[3-(morpholine-4-carbonyl)-[1,4′]bipiperidinyl-1′-yl]-methanone; (3R)-1′-(Anthracene-9-carbonyl)-[1,4′]bipiperidinyl-3-carboxylic acid diisopropylamide; (3R)-1′-(Anthracene-9-carbonyl)-[1,4′]bipiperidinyl-3-carboxylic acid (2,2,2-trifluoro-ethyl)-amide; (3R)-[7-chloro-2-(4-Chloro-phenyl)-6-methyl-quinolin-4-yl]-[3-(morpholine-4-carbonyl)-[1,4′]bipiperidinyl-1′-yl]-methanone; (3R)-1′-(Anthracene-9-carbonyl)-[1,4′]bipiperidinyl-3-carboxylic acid cyclohexyl-isopropyl-amide; (3R)-1′-(Anthracene-9-carbonyl)-[1,4′]bipiperidinyl-3-carboxylic acid cyclohexyl-ethyl-amide; (3R)-Anthracen-9-yl-[3-(6-fluoro-2-methyl-3,4-dihydro-2H-quinoline-1-carbon yl)-[1,4′]bipiperidinyl-1′-yl]-methanone; (3R)-1′-(Anthracene-9-carbonyl)-[1,4′]bipiperidinyl-3-carboxylic acid cyclobutylamide; (3R)-(2,6-Diphenyl-pyridin-4-yl)-[3-(morpholine-4-carbonyl)-[1,4′]bipiperidinyl-1′-yl]-methanone; (3R)-4-[1′-(Anthracene-9-carbonyl)-[1,4]bipiperidinyl-3-carbonyl]-piperazine-1-carboxylic acid dimethylamide; (cis-)(1′S,3′R)-anthracen-9-yl-{4-[3-(morpholine-4-carbonyl)-cyclohexyl]-piperazin-1-yl}-methanone; (3R)-[1′-(2,6-Diphenyl-pyridine-4-carbonyl)-[1,4′]bipiperidinyl-3-yl]-(2-ox a-5-aza-bicyclo[2.2.1]hept-5-yl)-methanone; (3R)-Anthracen-9-yl-[3-(meso-3,5-dimethyl-morpholine-4-carbonyl)-[1,4′]bipiperidinyl-1′-yl]-methanone; (3R)-Anthracen-9-yl-[3-(3R,5R-dimethyl-morpholine-4-carbonyl)-[1,4′]bipiperidinyl-1′-yl]-methanone; Anthracen-9-yl-[3-(morpholine-4-sulfonyl)-[1,4′]bipiperidinyl-1′-yl]-methanone (3R)-Anthracen-9-yl-[3-(3S,5S-dimethyl-morpholine-4-carbonyl)-[1,4′]bipiperidinyl-1′-yl]-methanone; (3R)-Anthracen-9-yl-{3-[3-(morpholine-4-carbonyl)-piperidin-1-yl]-azetidin-1-yl}-methanone; or pharmaceutically acceptable salts of said compounds. In one embodiment, the present invention includes the compound shown in Formula II, the

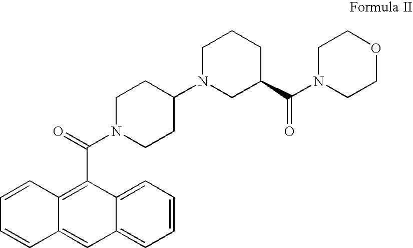

exemplary compound [(3R)-1-[1-(anthracene-9-carbonyl)piperidin-4-yl]piperidin-3-yl]-morpholin-4-ylmethanone (also called (R)-anthracen-9-yl(3-(piperidine-1-carbonyl)-1,4′-bipiperidin-1′-yl)methanone herein) (CP-640186). CP-640186 and many other compounds of this class are potent, reversible, isozyme-nonselective inhibitors of the CT (carboxyltranferase) reaction, interacting within the enzyme active center in a region near the binding site for the carboxy biotin moiety. Other exemplary compounds of this class which are suitable for use in the present invention include CP-497485, CP-610431, and/or CP-610432, as shown; (CP-610432 (S-1′-(anthracene-9-carbonyl)-N,N-diethyl-1,4′-bipiperidine-3-carboxamide); CP-610431 (R-1′-(anthracene-9-carbonyl)-N,N-diethyl-1,4′-bipiperidine-3-carboxamide); CP-497485 (1′-(anthracene-9-carbonyl)-N,N-diethyl-1,4′-bipiperidine-3-carboxamide)).

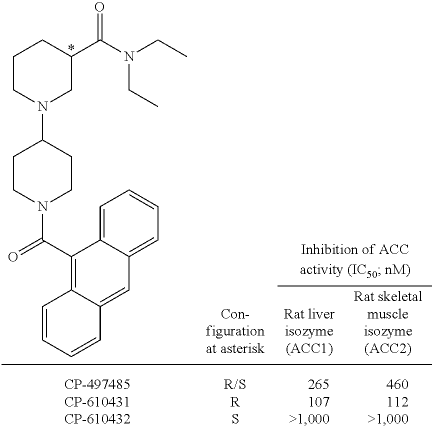

In one embodiment, a suitable ACC inhibitor is one or more of a class of compounds for treating or preventing loss of cognitive function caused by reduced neuronal metabolism which include bi-substrate analog CT inhibitors prepared by, for example, linking biotin to CoA via an acyl bridge between the thiol of CoA and the 1′-N of biotin and several series of acylsulfonamides. Exemplary compounds of this type include 1′-N-Chloroacetamido-biotin, benzyl ester (CABI); phenylmethyl 5-(1-{[(2-{[N-(2,4-dihydroxy-3,3-dimethylbutanyl)-5-(6-aminooctahydro-9H-purin-9-yl)-4-(hydroxyl-2-[(phosphonooxy)tetrahydrofuran-2-yl]methyl dihydrogen diphosphate-β-alanyl]amino}ethyl)thio]acetyl}-2-oxohexahydro-1H-thieno[3,4-d]imidazol-4-yl)pentanoate. Compound III (as well as Compound IV, below) is described in U.S. Pat. No. 6,485,941 which is incorporated by reference herein in its entirety, and is also incorporated with respect to Compounds III and IV and methods of manufacture.

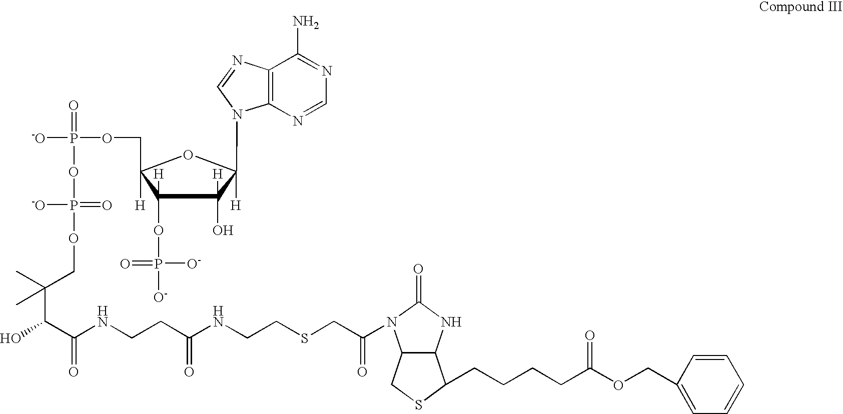

Another exemplary compound of this type includes Compound IV, see below.

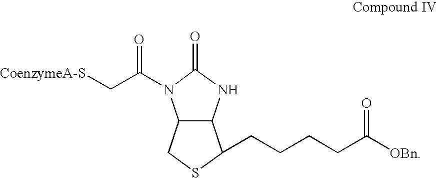

In one embodiment, a suitable ACC inhibitor is one or more of a class of compounds for treating or preventing loss of cognitive function caused by reduced neuronal metabolism which includes compounds of Formula V. Formula V is described in US Patent Application Publication No. 2006/0178400 which is incorporated by reference herein in its entirety. US Patent Application Publication No. 2006/0178400 is also incorporated with respect to the genus Formula V and all species described therein, as well as all methods of manufacturing described therein and shown below and includes compounds of formula V:

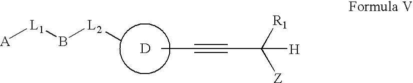

prodrugs thereof, or pharmaceutically acceptable salts of said compounds or of said prodrugs; wherein

A is selected from the group consisting of alkenyl, alkoxyalkyl, alkyl, aryl, arylalkyl, cycloalkyl, cycloalkylalkyl, haloalkyl, heteroaryl, heteroarylalkyl, heterocycle, and heterocyclealkyl;

B is selected from the group consisting of an aryl ring and a heteroaryl ring;

D is selected from the group consisting of an aryl ring and a heteroaryl ring;
L₁ is absent or is selected from the group consisting of hydroxyalkylene, —C(RₐR_{b})—, —C(O)—, —C(O)O—, —C(O)NH—, —NR_{c}—, —NR_{c}CH₂—, —NR_{c}C(O)—O—, —NH—N.dbd.CH—, —NR_{c}S(O)₂—, —O—, —OC(O)NH—, —OC(O)—, —O—N.dbd.CH—, —S—, —S(O)₂—, —S(O)₂NH—;
L₂ is selected from the group consisting of —C(R_{d}Rₑ)—, —(CH₂)ₙ—, —NH—, —O—, and —S—;
n is 1, 2 or 3;
Z is a member selected from the group consisting of alkoxy, hydroxy, hydroxyalkyl, R_{g}—O— and Rⱼ—NH—;
R₁ is hydrogen, C₁₋₆ haloalkyl or C₁₋₆ alkyl; Rₐ and R_{b} are each individually selected from the group consisting of hydrogen, alkyl, haloalkyl and hydroxy or Rₐ and R_{b} taken together with the atom to which they are attached form R_{f}N.dbd.;
R_{c} is selected from the group consisting of hydrogen, alkyl, aryl, haloalkyl, and heteroaryl;
R_{d} is selected from the group consisting of alkyl, haloalkyl, hydroxy and halo;
Rₑ is selected from the group consisting of hydrogen, alkyl, haloalkyl, hydroxy and halo, or R_{d} and Rₑ taken together with the atom to which they are attached form oxo;
R_{f} is selected from the group consisting of alkoxy, aryloxy, heteroaryloxy and hydroxy;
R_{g} is H₂N—C(O)— or C₁₋₆ alkylHN—C—(O)—; and
Rⱼ is a member selected from the group consisting of alkylcarbonyl, alkyl-NH—C(O)—, alkoxyalkyl, alkoxyalkylcarbonyl, alkoxycarbonyl, alkoxycarbonyl-NH-alkyl-NHC(O)—, alkoxy-NH—C(O)—, cyanoalkylcarbonyl, hydroxy, HONH—C(O)—, H₂NC(O)—, H₂NC(.dbd.NH)—, H₂NC(O)alkyl-NHC(O)—, H₂N—O—C(O)—, heteroaryl, heteroarylcarbonyl, heterocycle, and heterocyclecarbonyl.

Exemplary compounds of Formula V include N-{3-[2-(4-Alkoxyphenoxy)thiazol-5-yl]-1-methylprop-2-ynyl}carboxy derivatives, in particular containing 4-(thiazol-5-yl)but-3-yn-2-amino motifs.

In one embodiment, a suitable ACC inhibitor is one or more of a class of compounds for treating or preventing loss of cognitive function caused by reduced neuronal metabolism which includes lipophilic fatty acid mimetics. Inhibitor actions by this class in some cases is dependent on their intracellular conversion to their CoA thioesters. Compounds in this class may inhibit ACC activity by competing with acetyl-CoA in the CT reaction of the enzyme. Exemplary compounds of this class include 5-(tetradecyloxy)-2-furan-carboxylic acid (TOFA) for the elevation of circulating ketone bodies and treatment, prevention, inhibition or alleviation of diseases associated with hypometabolism including prodrugs thereof, or pharmaceutically acceptable salts of said compounds or of said prodrugs.

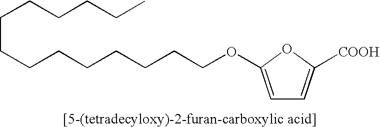

Other compounds of this class include the aryloxyphenoxypropionate and cyclohexanedione herbicides, such as haloxyfop and sethoxydim; MEDICA 16 (β,β,β′,β′-tetramethylhexadecanoic acid), ESP-55016 (8-hydroxy-2,2,14,14-tetra-methylpentadecanediotic acid), and S2E ((+)-p-[1-p-tert-butylphenyl)-2-oxo-4-pyrrolidinyl]methoxybenzoic acid).

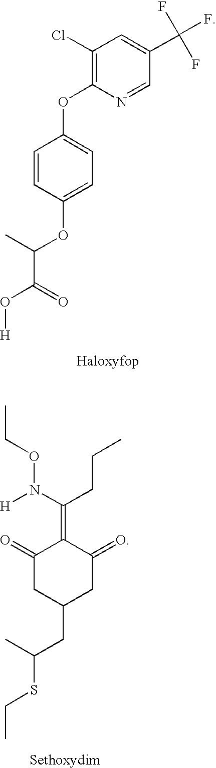

MEDICA 16 (β,β,β′,β′-tetramethylhexadecanoic acid),

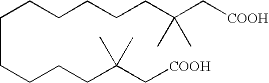

ESP-55016 (8-hydroxy-2,2,14,14-tetramethylpentadecanediotic acid), and S2E ((p)-p-[1-p-tert-butylphenyl)-2-oxo-4-pyrrolidinyl]methoxybenzoic acid).

Another compound of this class is AC-0417-9 (3,3-Dimethylhexanoate, monoglyceride) of the formula:

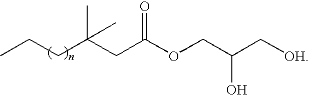

wherein n is 1-50 carbons. This compound leads to elevation of serum ketone levels when orally administered to mice (see Examples).

In one embodiment, a suitable ACC inhibitor is one or more of a class of compounds for treating or preventing loss of cognitive function caused by reduced neuronal metabolism which includes a class of ACC inhibitors known as the polyketide natural product fungicides, such as, for example, Soraphen A and derivatives. These compounds may inhibit activity by interacting with the enzyme at the allosteric site and interfering with the dimerization/oligomerization of the biotin carboxylase (BC) domain. Another preferred embodiment is the use of soraphen A and soraphen derivatives as described in US Patent Application Publication 2003/0144345, which is incorporated herein by reference in its entirety; this application is also incorporated specifically for the description and manufacturing of soraphen A and derivatives, including prodrugs thereof, or pharmaceutically acceptable salts of said compounds or of said prodrugs. Soraphen A ((1S,2S,3E,5R,6S,11S,14S,15R,16R,17S,18S)-15,17-dihydroxy-5,6,16-trimethoxy-2,14,18-trimethyl-11-phenyl-12,19-dioxabicyclo[13.3.1]nonadec-3-en-13-one) has the following formula:

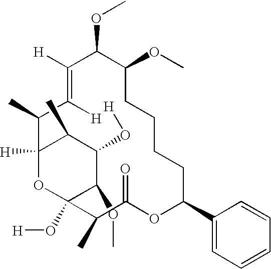

The present invention includes use of soraphen derivatives of formula (I)

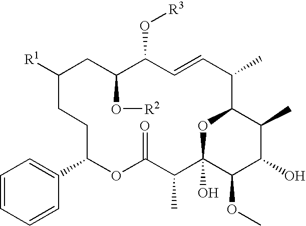

wherein
R₁ is hydrogen or hydroxy,
R² is hydrogen or lower-alkyl,
R³ is hydrogen or lower-alkyl,
In one embodiment, formula I is Soraphen A 1α., which is a compound of formula:

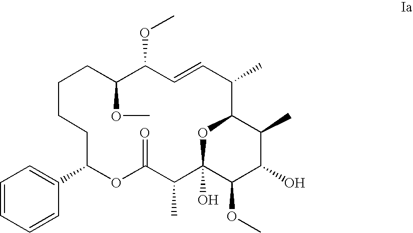

and pharmaceutically acceptable esters thereof.
Another embodiment includes Soraphen A 4-α, which is a compound of formula (I) as described above wherein R¹, R² and R³ are hydrogen. A further embodiment of the present invention is a compound of formula:

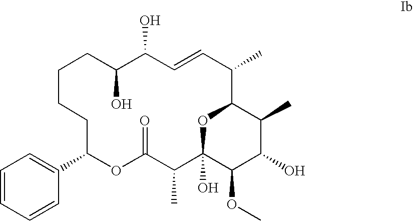

and pharmaceutically acceptable esters thereof.
Another compound is Soraphen B 2-α, which is the following formula:

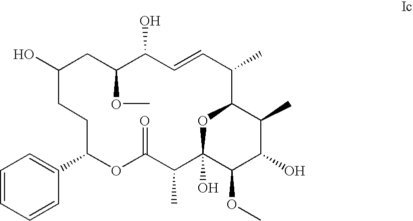

and pharmaceutically acceptable esters thereof.

In one embodiment, a suitable ACC inhibitor is one or more of a class of compounds for treating or preventing loss of cognitive function caused by reduced neuronal metabolism which includes a class of ACC inhibitors known as short interfering nucleic acid (siNA) molecules for modulating acetyl-CoA carboxylase gene expression, including decreasing expression (such as described in US Patent Application 2005/0124568, which is incorporated by reference herein in its entirety, and is also incorporated by reference specifically with respect to all species of short interfering nucleic acid (siNA), short interfering RNA (siRNA), double-stranded RNA (dsRNA), micro-RNA (miRNA), and short hairpin RNA (shRNA) molecules disclosed therein including all sequences of the same) for the elevation of circulating ketone bodies and treatment, prevention, inhibition or alleviation of diseases associated with hypometabolism. RNA interference refers to the process of sequence-specific post-transcriptional gene silencing in animals mediated by short interfering RNAs (siRNAs). In one embodiment, the invention includes methods for downregulation of acetyl-CoA carboxylase genes, using short interfering nucleic acid (siNA) molecules for the treatment or prevention of loss of cognitive function associated with decreased neuronal metabolism, as described elsewhere herein. Also included are compounds, compositions, and methods useful for modulating the expression and activity of other genes involved in pathways of acetyl-CoA carboxylase gene expression and/or activity by RNA interference (RNAi) using small nucleic acid molecules. In particular, the instant invention features small nucleic acid molecules, such as short interfering nucleic acid (siNA), short interfering RNA (siRNA), double-stranded RNA (dsRNA), micro-RNA (miRNA), and short hairpin RNA (shRNA) molecules and methods used to modulate the expression of acetyl-CoA carboxylase genes, such as acetyl-CoA carboxylase 1 and/or acetyl-CoA carboxylase 2.

In one embodiment, a suitable ACC inhibitor is one or more of a class of compounds for treating or preventing loss of cognitive function caused by reduced neuronal metabolism which includes compounds described in US Patent Application Publication No. 2007/0225332 for the treatment and prevention of diseases associated with neuronal hypometabolism. US Patent Application Publication No. 2007/0225332 is incorporated herein by reference in its entirety, and description of the genus shown below, all species, as well as methods of manufacture of the same, are specifically incorporated by reference.

The present invention is directed to compounds of formula VI,

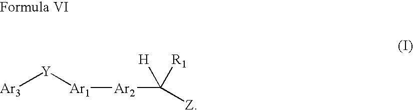

or a pharmaceutically acceptable salt, prodrug, salt of a prodrug, or combination thereof, wherein
R₁ is selected from the group consisting of hydrogen, cycloalkyl, alkyl and haloalkyl;
Y is selected from the group consisting of —(CR₄ₐR_{4b})ₘ—, —C(O)—, —O—, —N(H)—, —N(alkyl)- and —S—; wherein
m is 1, 2 or 3;
each of R₄ₐ, R_{4b}, at each occurrence, is independently selected from the group consisting of hydrogen, alkyl, hydroxyalkyl, and haloalkyl when m is 1, 2 or 3;
alternatively, R₄ₐ and R_{4b} together with the carbon to which they are attached form a monocyclic cycloalkyl or heterocycle ring when m is 1;
Ar₃ is phenyl or monocyclic heteroaryl; wherein Ar₃ is substituted with 1, 2 or 3 or 4 substituents independently selected from the group consisting of alkyl, alkenyl, —CN, —NO₂, halogen, —OR₅, —O—N═CH(R₂), —OC(O)R₂, —OC(O)N(R₃)(R₅), —OC(O)OR₂, —OS(O)₂R₅, —SR₂, —S(O)R₂, —S(O)₂R₅, —S(O)₂OR₅, —S(O)₂N(R₃)(R₅), —C(O)R₅, —C(O)N(R₃)(R₅), —C(O)OR₅, —C(O)N(R₃)(R₅), —N(R₃)(R₅), —N(H)—N═CH(R₂), —N(R₃)C(O)R₂, —N(R₃)C(O)OR₅, —N(R₃)S(O)₂R₅, —N(R₃)_{C}(O)_{N}(R₃)(R₅), —N(R₃)S(O)₂N(R₃)(R₅), —R₈ haloalkyl, cyanoalkyl, nitroalkyl, hydroxyalkyl, alkoxyalkyl, haloalkoxyalkyl, -alkylenyl-OC(O)R₂, -alkylenyl-OC(O)N(R₃)(R₅), -alkylenyl-OC(O)OR₂, -alkylenyl-OS(O)₂R₅, -alkylenyl-SR₂, -alkylenyl-S(O)R₂, -alkylenyl-S(O)₂R₅, -alkylenyl-S(O)₂OR₅, -alkylenyl-S(O)₂N(R₃)(R₅), -alkylenyl-C(O)R₅, -alkylenyl-C(O)N(R₃)(R₅), -alkylenyl-C(O)OR₅, -alkylenyl-C(O)N(R₃)(R₅), -alkylenyl-N(R₃)(R₅), -alkylenyl-N(R₃)C(O)R₂, -alkylenyl-N(R₃)C(O)OR₅-alkylenyl-N(R₃) S(O)₂R₅, -alkylenyl-N(R₃)C(O)N(R₃)(R₅), -alkylenyl-N(R₃)S(O)₂N(R₃)(R₅), and -alkylenyl-R₈;
R₂, at each occurrence, is independently selected from the group consisting of alkyl, alkenyl, haloalkyl, alkoxyalkyl, haloalkoxyalkyl, —R₈, and -alkylenyl-R₈;
R₃, at each occurrence, is independently selected from the group consisting of hydrogen, alkyl, arylalkyl, haloalkyl, and heteroarylalkyl;
R₅, at each occurrence, is independently selected from the group consisting of hydrogen, alkyl, alkenyl, haloalkyl, alkoxyalkyl, haloalkoxyalkyl, —R⁸, and -alkylenyl-R₈;
Ar₁ is selected from the group consisting of phenyl and a monocyclic, five or six-membered heteroaryl; —Ar₂ is a monocyclic five membered heteroaryl, wherein each Ar₂ is independently unsubstituted or substituted with 1 or 2 substituents selected from the group consisting of alkyl, alkenyl, halogen, —CN, —NO₂, hydroxy, alkoxy, —NH₂, —N(H)(alkyl), —N(alkyl)₂, —C(O)OH, —C(O)Oalkyl, —C(O)H, —C(O)alkyl, and haloalkyl;
Z is selected from the group consisting of —OR₉ₐ, -alkylenyl-OR₉ₐ, —NR₆R_{9b} and -alkylenyl-NR₆R_{9b}
R₆, at each occurrence, is independently selected from the group consisting of hydrogen, alkyl and haloalkyl;
R₉ₐ, at each occurrence, is independently selected from the group consisting of hydrogen, alkyl, haloalkyl, R₈, —C(O)OR₁₀, —S(O)₂R₁₀, —C(O)NR₇R₁₁, —S(O)₂NR₇R₁₁, —C(O)R₁₀, -alkylenyl-OR₁₀, -alkylenyl-NR₇R₁₁, -alkylenyl-N(R₇)C(O)OR₁₀, -alkylenyl-N(R₇)C(O)R₁₀, -alkylenyl-C(O)OR₁₀, -alkylenyl-S(O)₂R₁₀, -alkylenyl-S(O)₂NR₇R₁₁, -alkylenyl-C(O)NR₇R₁₁, -alkylenyl-C(O)R₁₀, and -alkylenyl-R₈,
R_{9b}, at each occurrence, is independently selected from the group consisting of hydrogen, alkyl, hydroxy, alkoxy, R₈, —C(═NH)NH₂, —C(O)OR₁₀, —S(O)₂R₁₀, —C(O)NR₇R₁₂, —C(O)ONH₂, —S(O)₂NR₇R₁₂, —C(O)R₁₀, —C(O)CH₂C(O)R₁₀, haloalkyl, -alkylenyl-OR₁₀, -alkylenyl-NR₇R₁₂, -alkylenyl-N(R₇)C(O)OR₁₀, -alkylenyl-N(R₇)C(O)R₁₀, -alkylenyl-C(O)OR₁₀, -alkylenyl-S(O)₂R₁₀, -alkylenyl-S(O)₂NR₇R₁₂, -alkylenyl-C(O)NR₇R₁₂, -alkylenyl-C(O)R₁₀, and -alkylenyl-R₈,
R₇, at each occurrence, are each independently selected from the group consisting of hydrogen, alkyl and haloalkyl;
R₁₀, at each occurrence, is independently selected from the group consisting of hydrogen, alkyl, alkoxyalkyl, cyanoalkyl, haloalkyl, —R₈, and alkylenyl-R₈;
R₁₁ at each occurrence, is independently selected from the group consisting of hydrogen, alkyl, hydroxy, alkoxy, alkoxyalkyl, cyanoalkyl, haloalkyl, —R₈, and -alkylenyl-R₈;
R₁₂, at each occurrence, is independently selected from the group consisting of hydrogen, alkyl, hydroxy, alkoxy, —R₈, alkoxyalkyl, cyanoalkyl, haloalkyl, -alkylenyl-C(O)NH₂, -alkylenyl-C(O)N(H)(alkyl), -alkylenyl-C(O)N(alkyl)₂, -alkylenyl-N(H)C(O)Oalkyl, -alkylenyl-N(alkyl)C(O)Oalkyl, and -alkylenyl-R₈; and
R₈, at each occurrence, is independently selected from the group consisting of aryl, heteroaryl, heterocycle, cycloalkyl and cycloalkenyl; and
the phenyl, cycloalkyl, cycloalkenyl, aryl, heteroaryl, heterocycle, aryl moiety of the arylalkyl, and the heteroaryl moiety of the heteroarylalkyl represented by Ar₁, R₃ and R₈, are each independently unsubstituted or substituted with 1, 2, 3 or 4 substituents independently selected from the group consisting of alkyl, alkenyl, —CN, —NO₂, halogen, ethylenedioxy, methylenedioxy, oxo, —ORₐ, —OC(O)Rₐ, —OC(O)ORₐ, —OS(O)₂Rₐ, —S(alkyl), —S(O)alkyl, —S(O)₂ alkyl, —S(O)₂ORₐ, —S(O)₂NRₐR_{b}, —C(O)ORₐ, —C(O)NRₐR_{b}, —C(O)ORₐ, —C(O)NRₐR_{b}, —NRₐR_{b}, —NORₐ, —N(R_{b})C(O)Rₐ, —N(R_{b})C(O)ORₐ, —N(R_{b})S(O)₂Rₐ, —N(R_{b})C(O)NRₐR_{b}, —N(R_{b})S(O)₂NRₐR_{b}, haloalkyl, cyanoalkyl, nitroalkyl, hydroxyalkyl, alkoxyalkyl, haloalkoxyalkyl, -alkylenyl-OC(O)Rₐ, -alkylenyl-OC(O)ORₐ, -alkylenyl-OS(O)₂ alkyl, -alkylenyl-S(alkyl), -alkylenyl-S(O)alkyl, -alkylenyl-S(O)₂ alkyl, -alkylenyl-S(O)₂ORₐ, -alkyl enyl-S(O)₂NRₐR_{b}, -alkylenyl-C(O)Rₐ, -alkylenyl-C(O)NRₐR_{b}, -alkylenyl-C(O)ORₐ, -alkylenyl-C(O)NRₐR_{b}, -alkylenyl-NRₐR_{b}, -alkylenyl-N(R_{b})C(O)Rₐ, -alkylenyl-N(R_{b}) C(O)OR^{a}, -alkylenyl-N(R_{b})S(O)₂Rₐ, -alkylenyl-N(R_{b})C(O)NRₐR_{b}, and -alkylenyl-N(R_{b})S(O)₂NRₐR_{b}; wherein Rₐ at each occurrence is independently selected from the group consisting of hydrogen, alkyl, alkenyl and haloalkyl, and R_{b} at each occurrence is independently selected from the group consisting of hydrogen and alkyl. The invention is also directed towards pharmaceutical compositions including the compounds of the present invention. Such compositions can be administered in accordance with methods of the present invention, typically as part of a therapeutic regimen for, treatment or prevention of conditions and disorders related to ACC. Another aspect of the present invention relates to a method of inhibiting ACC activity.

In one embodiment, a suitable ACC inhibitor is one or more of a class of compounds for treating or preventing loss of cognitive function caused by reduced neuronal metabolism which includes one or more compounds described in U.S. Pat. No. 5,602,164, which is incorporated by reference herein in its entirety, and also incorporated specifically with respect to compounds comprising lipophilic derivatives of natural amino acids and methods of making the same, having the general formula (I): R₄—(CH₂)ₙ—CO—N(R₁)—CH(R₂)—CO(—R₃), wherein R₁ represents H or CH₃; R₂ represents a side chain of a naturally occurring amino acid;

R₃ represents OH, OCH₂ CH₃ and NH₂; n is 6-18; and R₄ represents CH₃ or a group having the general formula (II): R₃—CO—CH(R₂)—N(R₁)—CO—, wherein R₁, R₂ and R₃ have the above meanings.

In one embodiment, a suitable ACC inhibitor is one or more of a class of compounds for treating or preventing loss of cognitive function caused by reduced neuronal metabolism which includes compounds described in U.S. Pat. No. 4,689,344 which have been found to be effective in blocking cholesterol and neutral lipid synthesis in-vivo without adversely affecting energy metabolism. U.S. U.S. Pat. No. 4,689,344 is incorporated herein by reference in its entirety, and is also incorporated by reference specifically with respect to the below generic formula and all species, including methods of manufacture of the same. In the present application they are found to be useful for the treatment, prevention, inhibition or alleviation of diseases associated with neuronal hypometabolism, such as Age Associated Memory Impairment (AAMI), Mild Cognitive Impairment (MCI), Alzheimer's disease, Parkinson's disease, Friedreich's Ataxia (FRDA), GLUT1-deficient Epilepsy, Leprechaunism and Rabson-Mendenhall Syndrome, Coronary Arterial Bypass Graft (CABG) dementia, anesthesia induced memory loss, Huntington's disease and many others.

The active compounds have the general formula

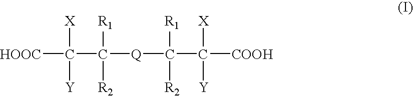

or in-vivo hydrolyzable functional derivatives of the carboxylic groups thereof, wherein
R₁ and R₂ each independently represent an unsubstituted or substituted hydrocarbyl or heterocyclyl radical;
X and Y each independently represents hydrogen, optionally substituted lower alkyl, halogen, cyano, carboxy, lower alkoxycarbonyl or carbamoyl; and
Q represents a diradical consisting of a linear chain of 8 to 14 carbon atoms, one or more of which may be replaced by heteroatoms, said chain being optionally substituted by inert substituents and one or more of said carbon or heteratom chain members optionally forming part of a ring structure.

The pharmaceutical compositions according to the invention are preferably in dosage unit form, each unit containing from 50 to 500 mg of the active ingredient of the formula (I) above. The daily dosage of the compounds of formula (I) above according to the invention will depend on the age, needs and tolerance of the individual patient, but will usually range from 50 mg to from 5000 mg per day.

In one embodiment, a suitable ACC inhibitor is one or more of a class of compounds for treating or preventing loss of cognitive function caused by reduced neuronal metabolism which includes pharmaceutical compositions as described in U.S. Pat. No. 4,908,385. U.S. Pat. No. 4,903,385 is specifically incorporated by reference herein in its entirety, and is also incorporated specifically with reference to the generic formula below, all species, and methods of manufacture of the same. Compounds are useful for the treatment, prevention, inhibition or alleviation of diseases associated with neuronal hypometabolism, such as Age Associated Memory Impairment (AAMI), Mild Cognitive Impairment (MCI), Alzheimer's disease, Parkinson's disease, Friedreich's Ataxia (FRDA), GLUT1-deficient Epilepsy, Leprechaunism and Rabson-Mendenhall Syndrome, Coronary Arterial Bypass Graft (CABG) dementia, anesthesia induced memory loss, Huntington's disease and many others, containing at least one α-halogenated dicarboxylic acid of the general formula:

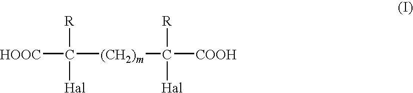

wherein Hal is a chlorine, bromine or fluorine atom, R is a hydrogen atom or Hal and m is a number of from 4 to 16, and/or at least one pharmacologically acceptable salt, ester or amide thereof.

### EXAMPLES

The following examples are offered by way of illustration and not by way of limitation.

### Example 1

### Mouse Pharmacokinetic (PK) Study

Purpose: Determine blood levels of ketone bodies after oral (po) and intraperitoneal (ip) dosing of TOFA at different time points in the mouse.
Animals: 75 ICR male mice 6 to 7 weeks old were used. Each mouse weighed between 20-30 grams.
General Study Design: Animals (housed 3/cage) were acclimated for at least 3 days prior to dosing. Mice were given either a single po dose (ranging from 0.5 mg/kg, to 5 mg/kg) of compound, or a single ip dose (1 mg/kg). Animals were anesthetized for blood collection at the times 0.5, 1, 2 and 3 hours. Whole blood (˜0.4 mls) was collected via cardiac puncture and collected into sodium heparin (Na Heparin, 1:9 ratio) anticoagulant. Blood was centrifuged for 8 minutes at 13,000 rpm to isolate plasma. The plasma was transferred into pre-labeled, color-coded eppendorf tubes and frozen at −70° C. Animals were observed for signs of toxicity and clinical observations recorded. Plasma levels of beta-hydroxybutyrate (BHB) were determined using a beta-hydroxybutyrate detection kit following manufacturer's directions (StanBio Inc.).
Materials: Compound, mouse gavage needles, vehicle, Isoflurane, 51 1 cc syringes w/26 g needles for blood collection, 63 eppendorf tubes for blood collection, 63 eppendorf tubes for plasma storage, centrifuge, container for sample storage, container for plasma shipment w/dry ice. AC-8632 (5-(tetradecyloxy)-2-furan-carboxylic acid (TOFA)) is a white crystalline solid, 100% active compound by weight. It is insoluble in aqueous solutions. It is soluble in ethanol (1 mg/ml), DMSO (2 mg/ml) and DMF (10 mg/ml). For maximum solubility in aqueous buffers, AC-8632 should first be dissolved in DMF and then diluted with the aqueous buffer of choice. AC-8632 was solubilized at a concentration of 0.5 mg/ml in a 1:1 solution of DMF::PBS (pH 7.2). For ip treatment, AC-8632 was mixed with DMSO at 2 mg/ml.
Results: AC-8632 (TOFA) elevated serum BHB levels at each dose from 0.5 mg-kg to 5 mg/kg. Levels of BHB continued to rise during the 3 hour time period with highest levels present at the 3 hour time point. Levels of BHB did not differ significantly by dose or by route of administration. Both po and ip dosing led to elevation of plasma BHB at the 3 hour time point. See Table 1. These results show that an oral dose of TOFA elevates the ketone body concentration in the blood.

TABLE 1
Mean BHB levels by dose and timepoint.

| Time | | n | Mean(mM) | Std Dev | Std Err | Treatment | Dose | | Method |
|---|---|---|---|---|---|---|---|---|---|
| 0 | hr | 3 | 0.135333 | 0.031501 | 0.018187 | AC-8632 | 0.5 | mg/kg | po |
| 0.5 | hr | 3 | 0.208667 | 0.004163 | 0.002404 | AC-8632 | 0.5 | mg/kg | po |
| 1 | hr | 3 | 0.236 | 0.038 | 0.021939 | AC-8632 | 0.5 | mg/kg | po |
| 2 | hr | 3 | 0.242667 | 0.131705 | 0.07604 | AC-8632 | 0.5 | mg/kg | po |
| 3 | hr | 3 | 0.347333 | 0.109038 | 0.062953 | AC-8632 | 0.5 | mg/kg | po |
| 0 | hr | 3 | 0.135333 | 0.031501 | 0.018187 | AC-8632 | 1 | mg/kg | po |
| 0.5 | hr | 3 | 0.209333 | 0.026633 | 0.015377 | AC-8632 | 1 | mg/kg | po |
| 1 | hr | 3 | 0.218667 | 0.008083 | 0.004667 | AC-8632 | 1 | mg/kg | po |
| 2 | hr | 3 | 0.282667 | 0.021502 | 0.012414 | AC-8632 | 1 | mg/kg | po |
| 3 | hr | 3 | 0.297333 | 0.070925 | 0.040948 | AC-8632 | 1 | mg/kg | po |
| 0 | hr | 3 | 0.135333 | 0.031501 | 0.018187 | AC-8632 | 2 | mg/kg | po |
| 0.5 | hr | 3 | 0.215333 | 0.081082 | 0.046813 | AC-8632 | 2 | mg/kg | po |
| 1 | hr | 3 | 0.149667 | 0.016289 | 0.009404 | AC-8632 | 2 | mg/kg | po |
| 2 | hr | 3 | 0.212667 | 0.032868 | 0.018977 | AC-8632 | 2 | mg/kg | po |
| 3 | hr | 3 | 0.339 | 0.14068 | 0.081222 | AC-8632 | 2 | mg/kg | po |
| 0 | hr | 3 | 0.135333 | 0.031501 | 0.018187 | AC-8632 | 5 | mg/kg | po |
| 0.5 | hr | 3 | 0.106 | 0.063695 | 0.036774 | AC-8632 | 5 | mg/kg | po |
| 1 | hr | 3 | 0.111 | 0.031241 | 0.018037 | AC-8632 | 5 | mg/kg | po |
| 2 | hr | 3 | 0.323 | 0.114503 | 0.066109 | AC-8632 | 5 | mg/kg | po |
| 3 | hr | 3 | 0.259 | 0.036592 | 0.021127 | AC-8632 | 5 | mg/kg | po |
| 0 | hr | 3 | 0.135333 | 0.031501 | 0.018187 | AC-8632 | 1 | mg/kg ip | ip |
| 0.5 | hr | 6 | 0.172333 | 0.048202 | 0.019679 | AC-8632 | 1 | mg/kg ip | ip |
| 1 | hr | 6 | 0.177 | 0.050064 | 0.020439 | AC-8632 | 1 | mg/kg ip | ip |
| 2 | hr | 6 | 0.209 | 0.084226 | 0.034385 | AC-8632 | 1 | mg/kg ip | ip |
| 3 | hr | 6 | 0.216333 | 0.101402 | 0.041397 | AC-8632 | 1 | mg/kg ip | ip |

### Example 2

Use of an ACC inhibitor to elevate serum ketone levels in a rat model Sprague-Dawley rats are fed a standard commercial rat chow. After 15 days of acclimation, two groups of rats are fed an experimental diet containing 1-50 mg/kg/day of an ACC inhibitor, such as 5-(tetradecyloxy)-2-furan-carboxylic acid (TOFA) or CP-610431. A control group is kept on the standard chow.

The weight of each rat is measured daily. Urine samples are collected daily and analyzed for 3-hydroxybutyrate by enzymatic assay. After 5 days on the experimental diet, the rats are euthanized, and a blood sample was collected and analyzed for 3-hydroxybutyrate, acetoacetate and acetone by standard enzymatic techniques.

The concentration of ketone bodies in the rat blood plasma collected at time of euthanasia is measured by enzymatic methods. The control group is expected to show normal concentrations of 3-hydroxybutyrate and acetoacetate, approximately, 0.02-0.07 mM. Rats fed the ACC inhibitor are expected to have elevated 3-hydroxybutyrate, acetoacetate and malate concentrations. These results show that rats fed ACC inhibitor had increased levels of ketone bodies in their blood.

The concentration of 3-hydroxybutyrate in the urine of rats fed TOFA or compound 1 is determined by GC-MS to be approximately 1-10 mM, respectively. 3-Hydroxybutyrate is undetectable in the urine of the control rats. These results show that an oral dose of TOFA or CP-610431 elevates the ketone body concentration in the blood and in the urine.

### Example 3

Neuroprotective effects of an ACC inhibitor in MPTP lesioned mice. 1-methyl-4-phenyl-1,2,3,6-tetrahydropyridine (MPTP), blocks complex I (NADH-ubiquinone oxidoreductase) of the mitochondrial electron transport chain, and causes typical symptoms of Parkinson's disease (PD) and the loss of dopaminergic neurons. C57BL6 mice receive daily orally gavage of CP-610431 at doses ranging from 0.001-5 mg/kg/day for seven days before they are lesioned with MPTP. During the 7 days of MPTP treatment animals will continue treatment with compound 1 or with a placebo. At the end of seven days animals are tested for behavioral effects of MPTP treatment and histopathology is performed.

Three groups of 10 animals are used for this experiment. Group 1 is treated with CP-610431 and lesioned with MPTP, Group 2 is treated with placebo and lesioned with MPTP, and Group 3 is treated with placebo and sham lesioned. After seven days of MPTP treatment behavior and motor skills are tested on all mice. Three tests are used to assess protective behavioral effects: Open Field behavior, Beam Walking test, and Rota Rod test. Improved behavior and motor skills are anticipated in animals treated with CP-610431.

Following behavioral and motor testing, brain tissue and blood is collected from the mice and sampled. An overview staining is performed. (Nissle) and

Dopamine histopathology is immunohistochemically evaluated comparing tyrosine-hydroxylase-positive (TH+) neurons in the substantia nigra density of TH+ nerve terminals in the striatum on the lesioned side of drug treated and control animals. Increased number of TH+ neurons are anticipated in the animals treated with CP-610431.

### Example 4

### Mouse Pharmacokinetic (PK) Study of AC-0417-9

Purpose: Determine blood levels of ketone bodies after oral dosing of AC-0417-9 (3,3-Dimethylhexanoate, monoglyceride) at different time points in the mouse.
Animals: 12 ICR male mice 6 to 7 weeks old were used. Each mouse weighed between 20-30 grams.
General Study Design: Animals (housed 3/cage) were acclimated for at least 3 days prior to dosing. Mice were given a single po dose of 3 g/kg. Animals were anesthetized for blood collection at the times 0.5, 1, 2 and 3 hours. Whole blood (˜0.4 mls) was collected via cardiac puncture and collected into sodium heparin (Na Heparin, 1:9 ratio) anticoagulant. Blood was centrifuged for 8 minutes at 13,000 rpm to isolate plasma. The plasma was transferred into pre-labeled, color-coded eppendorf tubes and frozen at −70° C. Animals were observed for signs of toxicity and clinical observations recorded. Plasma levels of beta-hydroxybutyrate (BHB) were determined using a beta-hydroxybutyrate detection kit following manufacturer's directions (StanBio Inc.).
Materials: Compound, mouse gavage needles, vehicle, Isoflurane, 1 cc syringes w/26 g needles for blood collection, eppendorf tubes for blood collection, eppendorf tubes for plasma storage, centrifuge, container for sample storage, container for plasma shipment w/dry ice. AC-0417-9 is a clear liquid, 100% active compound by weight.
Results: AC-0417-9 was demonstrated to elevate serum BHB levels. Levels of BHB continued to rise during the 3 hour time period with highest levels present at the 3 hour time point.

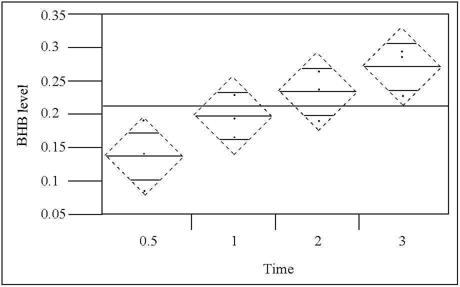

These results show that an oral dose of AC-0417-9 elevates the ketone body concentration in the blood.

### Example 5

To evaluate the safety, tolerability and effectiveness of CP-610431 in Alzheimer's disease.

CP-610431 is administered once a day for ninety days in subjects with mild to moderate, probable Alzheimer's disease. A randomized, double-blind, placebo-controlled, parallel, multi-center design is used. Following a screening period of up to four weeks, subjects will receive either TOFA or placebo for ninety days followed by a two-week washout period.

Study subjects are 100 outpatients diagnosed as having probable Alzheimer's disease of mild to moderate severity. During the double-blind period of the protocol, 50 subjects receive active medication, and 50 subjects receive placebo.

CP-610431 or matching placebo will be administered once a day for ninety days. Following the end of the ninety-day dosing period, subjects will have a two-week study medication washout period. Each subject is seen five (5) times: at Screening, Baseline, and post-baseline Days 45, 90, and 104. Adverse events, vital signs, weight, physical examinations, 12-lead ECGs, laboratory tests are examined. Primary outcome measures are: Alzheimer's Disease Assessment Scale—Cognitive Subscale (ADAS-Cog), Alzheimer's Disease Cooperative Study—Clinician's Global Impression of Change (ADCS-CGIC) and the Mini-Mental State Examination (MMSE). It is anticipated that subjects treated with compound III will show improvement in one or more outcome measures, including ADAS-Cog, ADCS-CGIC or MMSE.

β-Hydroxybutyrate levels are measured pre-dose and 2 hr post-dose on Day 0 (Baseline), Day 45, and Day 90. β-Hydroxybutyrate Cmin levels are also measured at Screening and at the conclusion of the washout period (Day 104). ApoE genotype will be measured on subjects that provide consent. It is anticipated that subjects treated with CP-610431 will show elevated serum ketone body levels.

All publications and patent applications cited in this specification are herein incorporated by reference as if each individual publication or patent application were specifically, and individually, indicated to be incorporated by reference.

While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings without departing from the essential scope thereof. Therefore, it is intended that the invention not be limited to the particular embodiment disclosed as the best mode contemplated for carrying out this invention, but that the invention will include all embodiments falling within the scope of the appended claims.

1. A method of treating loss of cognitive function caused by reduced neuronal metabolism, wherein said treatment comprises administration of a pharmaceutical composition comprising a compound capable of inhibiting Acetyl CoA Carboxylase to a patient in need thereof in an amount sufficient to cause hyperketonemia in the patient, resulting in ketone bodies being utilized for energy in the brain.

2. The method of claim 1, wherein the blood level of D-beta-hydroxybutyrate in the patient is raised to about 0.1 to 50 mM.

3. The method of claim 1, wherein the blood level of D-beta-hydroxybutyrate is raised to about 0.2 to 20 mM.

4. The method of claim 1, wherein the blood level of D-beta-hydroxybutyrate is raised to about 0.3 to 5 mM.

5. The method of claim 1, wherein the blood level of D-beta-hydroxybutyrate is raised to about 0.5 to 2 mM.

6. The method of claim 1, wherein the blood level of D-beta-hydroxybutyrate is raised to about 1 to 10 mM.

7. The method of claim 1, wherein the loss of cognitive function is caused by Alzheimer's Disease or Mild Cognitive Impairment.

8. The method of claim 1, wherein the patient's apolipoprotein E genotype is APOE4 (−).

9. The method of claim 1, wherein the pharmaceutical composition causes hyperketonemia in the patient when the patient has a diet wherein carbohydrate intake is not restricted.

10. The method of claim 1, wherein the acetyl CoA carboxylase inhibitor is selected from the group consisting of
[(3R)-1-[1-(anthracene-9-carbonyl)piperidin-4-yl]piperidin-3-yl]-morpholin-4-ylmethanone (CP 640186),
CP-610432 (S-1′-(anthracene-9-carbonyl)-N,N-diethyl-1,4′-bipiperidine-3-carboxamide);
CP-610431 (R-1′-(anthracene-9-carbonyl)-N,N-diethyl-1,4′-bipiperidine-3-carboxamide);
CP-497485 (1′-(anthracene-9-carbonyl)-N,N-diethyl-1,4′-bipiperidine-3-carboxamide);
phenylmethyl 5-(1-{[(2-{[N-(2,4-dihydroxy-3,3-dimethylbutanyl)-5-(6-aminooctahydro-9H-purin-9-yl)-4-(hydroxyl-2-[(phosphonooxy)tetrahydrofuran-2-yl]methyl dihydrogen diphosphate-β-alanyl]amino}ethyl)thio]acetyl}-2-oxohexahydro-1H-thieno[3,4-d]imidazol-4-yl)pentanoate,
5-(tetradecyloxy)-2-furan-carboxylic acid (TOFA),
3,3-dimethylhexanoate, monoglyceride (AC-0417-9),
MEDICA 16 (β,β,β′,β′-tetramethylhexadecanoic acid),
ESP-55016 (8-hydroxy-2,2,14,14-tetra-methylpentadecanediotic acid),
S2E ((+)-p-[1-p-tert-butylphenyl)-2-oxo-4-pyrrolidinyl]methoxybenzoic acid);
1S,2S,3E,5R,6S,11S,14S,15R,16R,17S,18S)-15,17-dihydroxy-5,6,16-trimethoxy-2,14,18-trimethyl-11-phenyl-12,19-dioxabicyclo[13.3.1]nonadec-3-en-13-one (Soraphen A); and
1′-N-Chloroacetamido-biotin, benzyl ester (CABI).

11. A method of selecting a patient for treatment with a compound capable of elevating ketone body concentrations, comprising:
a) selecting a patient having loss of cognitive function caused by reduced neuronal metabolism; and
b) determining a patient's apolipoprotein E genotype; and
c) providing a pharmaceutical composition comprising an acetyl CoA carboxylase inhibitor to a patient having an absence of APOE4 in an amount effective for the treating loss of cognitive function caused by reduced neuronal metabolism.

12. The method of claim 11 wherein the genotype is determined to be APOE4(−).

13. The method of claim 11, wherein the blood level of D-beta-hydroxybutyrate in the patient is raised to about 0.1 to 50 mM.

14. The method of claim 11, wherein the blood level of D-beta-hydroxybutyrate is raised to about 0.2 to 20 mM.

15. The method of claim 1, wherein the blood level of D-beta-hydroxybutyrate is raised to about 0.3 to 5 mM.

16. The method of claim 11, wherein the blood level of D-beta-hydroxybutyrate is raised to about 0.5 to 2 mM.

17. The method of claim 11, wherein the blood level of D-beta-hydroxybutyrate is raised to about 1 to 10 mM.

18. The method of claim 11, wherein the loss of cognitive function is caused by Alzheimer's Disease or Mild Cognitive Impairment.

19. The method of claim 11, wherein the pharmaceutical composition causes hyperketonemia in the patient when the patient has a diet wherein carbohydrate intake is not restricted.

20. The method of claim 1, wherein the acetyl CoA carboxylase inhibitor is selected from the group consisting of
[(3R)-1-[1-(anthracene-9-carbonyl)piperidin-4-yl]piperidin-3-yl]-morpholin-4-ylmethanone (CP 640186);
CP-610432 (S-1′-(anthracene-9-carbonyl)-N,N-diethyl-1,4′-bipiperidine-3-carboxamide);
CP-610431 (R-1′-(anthracene-9-carbonyl)-N,N-diethyl-1,4′-bipiperidine-3-carboxamide);
CP-497485 (1′-(anthracene-9-carbonyl)-N,N-diethyl-1,4′-bipiperidine-3-carboxamide);
phenylmethyl 5-(1-{[(2-{[N-(2,4-dihydroxy-3,3-dimethylbutanyl)-5-(6-aminooctahydro-9H-purin-9-yl)-4-(hydroxyl-2-[(phosphonooxy)tetrahydrofuran-2-yl]methyl dihydrogen diphosphate-β-alanyl]amino}ethyl)thio]acetyl}-2-oxohexahydro-1H-thieno[3,4-d]imidazol-4-yl)pentanoate;
5-(tetradecyloxy)-2-furan-carboxylic acid (TOFA);
3,3-dimethylhexanoate, monoglyceride (AC-0417-9);
MEDICA 16 (β,β,β′,β′-tetramethylhexadecanoic acid);
ESP-55016 (8-hydroxy-2,2,14,14-tetra-methylpentadecanediotic acid),
S2E ((+)-p-[1-p-tert-butylphenyl)-2-oxo-4-pyrrolidinyl]methoxybenzoic acid);
1S,2S,3E,5R,6S,11S,14S,15R,16R,17S,18S)-15,17-dihydroxy-5,6,16-trimethoxy-2,14,18-trimethyl-11-phenyl-12,19-dioxabicyclo[13.3.1]nonadec-3-en-13-one (Soraphen A); and
1′-N-Chloroacetamido-biotin, benzyl ester (CABI).
